SEQUENCE LISTING (10 sequences)

SEQ ID NO: 1 - protein, 796 aa, Homo sapiens
  tropomyosin receptor kinase A, TrkA
  misc_feature: NCBI Accession Number NP_002520.2
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 2 - protein, 277 aa, Homo sapiens
  kinase domain of tropomyosin receptor kinase A
  misc_feature: fragment of the polypeptide sequence at SEQ ID NO 1
HIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHAR

SEQ ID NO: 3 - protein, 838 aa, Homo sapiens
  tropomyosin receptor kinase B, TrkB
  misc_feature: NCBI Accession Number NP_006171.2
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 4 - protein, 275 aa, Homo sapiens
  kinase domain of tropomyosin receptor kinase B
  misc_feature: fragment of the polypeptide sequence at SEQ ID NO 3
HIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTL

SEQ ID NO: 5 - protein, 839 aa, Homo sapiens
  tropomyosin receptor kinase C, TrkC
  misc_feature: NCBI Accession Number NP_001012338.1
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 6 - protein, 292 aa, Homo sapiens
  kinase domain of tropomyosin receptor kinase C
  misc_feature: fragment of the polypeptide sequence at SEQ ID NO 5
HIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKI

SEQ ID NO: 7 - protein, 1620 aa, Homo sapiens
  ALK tyrosine kinase receptor, ALK
  misc_feature: NCBI Accession Number NP_004295
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 8 - protein, 273 aa, Homo sapiens
  kinase domain of ALK tyrosine kinase
  misc_feature: fragment of the polypeptide sequence at SEQ ID NO 7
EVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILER

SEQ ID NO: 9 - protein, 2347 aa, Homo sapiens
  proto-oncogene tyrosine-protein kinase ROS, ROS1
  misc_feature: NCBI Accession Number NP_002935
MKNIYCLIPKLVNFATLGCLWISVVQCTVLNSCLKSCVTNLGQQLDLGTPHNLSEPCIQGCHFWNSVDQKNCALKCRESCEVGCSSAEGAYEEEVLENADLPTAPFASSIGSHNMTLRWKSANFSGVKYIIQWKYAQLLGSWTYTKTVSRPSYVVKPLHPFTEYIFRVVWIFTAQLQLYSPPSPSYRTHPHGVPETAPLIRNIESSSPDTVEVSWDPPQFPGGPILGYNLRLISKNQKLDAGTQRTSFQFYSTLPNTIYRFSIAAVNEVGEGPEAESSITTSSSAVQQEEQWLFLSRKTSLRKRSLKHLVDEAHCLRLDAIYHNITGISVDVHQQIVYFSEGTLIWAKKAANMSDVSDLRIFYRGSGLISSISIDWLYQRMYFIMDELVCVCDLENCSNIEEITPPSISAPQKIVADSYNGYVFYLLRDGIYRADLPVPSGRCAEAVRIVESCTLKDFAIKPQAKRIIYFNDTAQVFMSTFLDGSASHLILPRIPFADVKSFACENNDFLVTDGKVIFQQDALSFNEFIVGCDLSHIEEFGFGNLVIFGSSSQLHPLPGRPQELSVLFGSHQALVQWKPPALAIGANVILISDIIELFELGPSAWQNWTYEVKVSTQDPPEVTHIFLNISGTMLNVPELQSAMKYKVSVRASSPKRPGPWSEPSVGTTLVPASEPPFIMAVKEDGLWSKPLNSFGPGEFLSSDIGNVSDMDWYNNSLYYSDTKGDVFVWLLNGTDISENYHLPSIAGAGALAFEWLGHFLYWAGKTYVIQRQSVLTGHTDIVTHVKLLVNDMVVDSVGGYLYWTTLYSVESTRLNGESSLVLQTQPWFSGKKVIALTLDLSDGLLYWLVQDSQCIHLYTAVLRGQSTGDTTITEFAAWSTSEISQNALMYYSGRLFWINGFRIITTQEIGQKTSVSVLEPARFNQFTIIQTSLKPLPGNFSFTPKVIPDSVQESSFRIEGNASSFQILWNGPPAVDWGVVFYSVEFSAHSKFLASEQHSLPVFTVEGLEPYALFNLSVTPYTYWGKGPKTSLSLRAPETVPSAPENPRIFILPSGKCCNKNEVVVEFRWNKPKHENGVLTKFEIFYNISNQSITNKTCEDWIAVNVTPSVMSFQLEGMSPRCFIAFQVRAFTSKGPGPYADVVKSTTSEINPFPHLITLLGNKIVFLDMDQNQVVWTFSAERVISAVCYTADNEMGYYAEGDSLFLLHLHNRSSSELFQDSLVFDITVITIDWISRHLYFALKESQNGMQVFDVDLEHKVKYPREVKIHNRNSTIISFSVYPLLSRLYWTEVSNFGYQMFYYSIISHTLHRILQPTATNQQNKRNQCSCNVTEFELSGAMAIDTSNLEKPLIYFAKAQEIWAMDLEGCQCWRVITVPAMLAGKTLVSLTVDGDLIYWIITAKDSTQIYQAKKGNGAIVSQVKALRSRHILAYSSVMQPFPDKAFLSLASDTVEPTILNATNTSLTIRLPLAKTNLTWYGITSPTPTYLVYYAEVNDRKNSSDLKYRILEFQDSIALIEDLQPFSTYMIQIAVKNYYSDPLEHLPPGKEIWGKTKNGVPEAVQLINTTVRSDTSLIISWRESHKPNGPKESVRYQLAISHLALIPETPLRQSEFPNGRLTLLVTRLSGGNIYVLKVLACHSEEMWCTESHPVTVEMFNTPEKPYSLVPENTSLQFNWKAPLNVNLIRFWVELQKWKYNEFYHVKTSCSQGPAYVCNITNLQPYTSYNVRVVVVYKTGENSTSLPESFKTKAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 10 - protein, 276 aa, Homo sapiens
  kinase domain of ROS1
  misc_feature: fragment of the polypeptide sequence at SEQ ID NO 9
AFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQ

(19) United States
(12) Patent Application Publication
(10) Pub. No.: US 2018/0177792 A1
(43) Pub. Date: Jun. 28, 2018

(54) COMPOSITIONS AND METHODS FOR TREATING PATIENTS WITH RTK MUTANT CELLS

(71) Applicant: IGNYTA, INC., San Diego, CA (US)

(72) Inventor: Ge WEI, San Diego, CA (US)

(73) Assignee: IGNYTA, INC., San Diego, CA (US)

(21) Appl. No.: 15/577,729

(22) PCT Filed: May 25, 2016
(86) PCT No.: PCT/US2016/034166

§ 371 Date: Nov. 28, 2017

Related U.S. Application Data
(60) Provisional application No. 62/168,237, filed on May 29, 2015.
(60) Provisional application No. 62/309,900, filed on Mar. 17, 2016.

(51) Int. Cl. A61K 31/553; A61P 35/00; C12Q 1/6886; A61K 31/496; A61K 31/519

(52) U.S. Cl. CPC A61K 31/553; A61P 35/00; C12Q 1/6886; C12Q 2600/106; A61K 31/519; C12Q 2600/156; A61K 31/496

(57) ABSTRACT

Disclosed herein are compositions and methods for treating cancer patients who have been previously treated with one or more chemotherapeutic agents and have developed at least partial resistance to such chemotherapeutic agents. Also disclosed are methods for selecting compounds suitable for treatment of cancer in a patient who has become resistant to an inhibitor of a receptor tyrosine kinase (RTK).

### RELATED APPLICATIONS

The present application claims priority to U.S. Provisional Patent Application Ser. No. 62/168,237, filed on May 29, 2015, and U.S. Provisional Patent Application Ser. No. 62/309,900, filed on Mar. 17, 2016. The contents of the above-referenced applications are hereby expressly incorporated by reference in their entireties.

### INCORPORATION OF THE SEQUENCE LISTING

The material in the accompanying sequence listing is hereby incorporated by reference into this application. The accompanying sequence listing text file, named IGNYT.051WO_Sequence Listing, was created on May 5, 2016 and is 69 KB. The file can be assessed using Microsoft Word on a computer that uses Windows OS.

### FIELD

The present disclosure relates to compositions and methods for treating cancer patients, for example cancer patients who have been previously treated with one or more chemotherapeutic agents and have developed at least partial resistance to the one or more chemotherapeutic agents.

### BACKGROUND

The materials described in this section are not admitted to be prior art by inclusion in this section.

Cancer chemotherapy, particularly with a combination of anti-cancer agents, has increasingly become the treatment of choice for delocalized tumors that are untreatable by surgery or radiation. However, in many cases the cancers acquire resistance to these chosen chemotherapeutics and ultimately become refractory to treatment. As a result, some patients relapse after even a short period of time, and do not respond to a second course of chemotherapy.

The underlying cause of progressive drug resistance is generally related to spontaneous genetic mutations which occur in all living cells, which mutations are inheritable and may be passed on to succeeding generations. In any cell population, including cancer cell populations, mutants that are resistant to any given drug occur at a frequency of somewhere between one in 10⁵ and one in 10⁸ cells. Although this is a very rare event, it can have a large impact on the outcome of chemotherapy.

Therefore, there is a need for the determining the underlying causes of such resistance so that suitable diagnostic tests can be developed and more effective treatments can be provided. Moreover, there is a need for new compounds that are able to treat patients that show cancer progression or relapse despite initial response to current tyrosine kinase inhibitors.

### SUMMARY

This section provides a general summary of the disclosure, and is not comprehensive of its full scope or all of its features.

In one aspect, disclosed herein are methods for treating cancer in a patient, comprising (a) acquiring knowledge of the presence of one or more molecular alterations in a biological sample from the patient, wherein the one or more molecular alterations includes one or more mutations in one or more receptor tyrosine kinase polypeptides selected from TrkA, TrkB, TrkC, ALK and ROS1; (b) selecting a chemotherapeutic agent appropriate for the treatment of the cancer; and (c) administering a therapeutically effective amount of the selected chemotherapeutic agent to the patient.

Implementations of the methods disclosed herein can include one or more of the following features. In some embodiments, the one or more mutation includes one or more amino acid substitutions in a kinase catalytic domain of the one or more receptor tyrosine kinase polypeptides. In some embodiments, the one or more one amino acid substitutions is at a position corresponding to an amino acid residue selected from the amino acid residues identified in FIG. 1 and/or TABLE 1 as conserved residues, and combinations of any thereof. In some embodiments, the one or more amino acid substitutions is at a position corresponding to an amino acid residue selected from V573, F589, G595 and G667 of the TrkA polypeptide of SEQ ID NO: 1; V619, F633, G639 and G709 of the TrkB polypeptide of SEQ ID NO: 3; V603, F618, G623 and G696 of the TrkC polypeptide of SEQ ID NO: 5; V1182, L1196, G1202 and G1269 of the ALK polypeptide of SEQ ID NO: 7; and L2012, L2026, G2032 and G2101 of the ROS1 polypeptide of SEQ ID NO: 9. In some embodiments, the one or more amino acid substitutions is at a position corresponding to amino acid residue V573 of the TrkA polypeptide of SEQ ID NO: 1. In some embodiments, the one or more amino acid substitutions is a Val-to-Met substitution at a position corresponding to amino acid residue V573 of the TrkA polypeptide (V573M). In some embodiments, the one or more amino acid substitutions is at a position corresponding to amino acid residue F589 of the TrkA polypeptide of SEQ ID NO: 1. In some embodiments, the one or more amino acid substitutions is a Phe-to-Leu substitution at a position corresponding to amino acid residue F589 of the TrkA polypeptide (F589L). In some embodiments, the one or more amino acid substitutions is at a position corresponding to amino acid residue G595 of the TrkA polypeptide of SEQ ID NO: 1. In some embodiments, the one or more amino acid substitutions is a Gly-to-Arg substitution at a position corresponding to amino acid residue G595 of the TrkA polypeptide (G595R). In some embodiments, the one or more amino acid substitutions is at a position corresponding to amino acid residue G667 of the TrkA polypeptide of SEQ ID NO: 1. In some embodiments, the one or more amino acid substitutions is a Gly-to-Cys substitution at a position corresponding to amino acid residue G667 of the TrkA polypeptide (G667C). In some embodiments, the one or more amino acid substitutions is a Gly-to-Ala substitution at a position corresponding to amino acid residue G667 of the TrkA polypeptide (G667A). In some embodiments, the one or more amino acid substitutions is a Gly-to-Ser substitution at a position corresponding to amino acid residue G667 of the TrkA polypeptide (G667S). In some embodiments, the one or more amino acid substitutions is at a position corresponding to amino acid residue V619 of the TrkB polypeptide of SEQ ID NO: 3. In some embodiments, the one or more amino acid substitutions is a Val-to-Met substitution at a position corresponding to amino acid residue V619 of the TrkB polypeptide (V619M). In some embodiments, the one or more amino acid substitutions is at a position corresponding to amino acid residue F633 of the TrkB polypeptide of SEQ ID NO: 3. In some embodiments, the one or more amino acid substitutions is a Phe-to-Leu substitution at a position corresponding to amino acid residue F633 of the TrkB polypeptide (F633L). In some embodiments, the one or more amino acid substitutions is at a position corresponding to amino acid residue G639 of the TrkB polypeptide of SEQ ID NO: 3. In some embodiments, the one or more amino acid substitutions is a Gly-to-Arg substitution at a position corresponding to amino acid residue G639 of the TrkB polypeptide (G639R). In some embodiments, the one or more amino acid substitutions is at a position corresponding to amino acid residue G709 of the TrkB polypeptide of SEQ ID NO: 3. In some embodiments, the one or more amino acid substitutions is a Gly-to-Cys substitution at a position corresponding to amino acid residue G709 of the TrkB polypeptide (G709C). In some embodiments, the one or more amino acid substitutions is a Gly-to-Ala substitution at a position corresponding to amino acid residue G709 of the TrkB polypeptide (G709A). In some embodiments, the one or more amino acid substitutions is a Gly-to-Ser substitution at a position corresponding to amino acid residue G709 of the TrkB polypeptide (G709S). In some embodiments, the one or more amino acid substitutions is at a position corresponding to amino acid residue V603 of the TrkC polypeptide of SEQ ID NO: 5. In some embodiments, the one or more amino acid substitutions is a Val-to-Met substitution at a position corresponding to amino acid residue V603 of the TrkC polypeptide of SEQ ID NO: 5 (V603M). In some embodiments, the one or more amino acid substitutions is at a position corresponding to amino acid residue F617 of the TrkC polypeptide of SEQ ID NO: 5. In some embodiments, the one or more amino acid substitutions is a Phe-to-Leu substitution at a position corresponding to amino acid residue F617 of the TrkC polypeptide of SEQ ID NO: 5 (F617L). In some embodiments, the one or more amino acid substitutions is at a position corresponding to amino acid residue G623 of the TrkC polypeptide of SEQ ID NO: 5. In some embodiments, the one or more amino acid substitutions is a Gly-to-Arg substitution at a position corresponding to amino acid residue G623 of the TrkC polypeptide (G623R). In some embodiments, the one or more amino acid substitutions is at a position corresponding to amino acid residue G696 of the TrkC polypeptide of SEQ ID NO: 5. In some embodiments, the one or more amino acid substitutions is a Gly-to-Cys substitution at a position corresponding to amino acid residue G696 of the TrkC polypeptide (G696C). In some embodiments, the one or more amino acid substitutions is a Gly-to-Ala substitution at a position corresponding to amino acid residue G696 of the TrkB polypeptide (G696A). In some embodiments, the one or more amino acid substitutions is a Gly-to-Ser substitution at a position corresponding to amino acid residue G696 of the TrkB polypeptide (G696S).

In some embodiments, the patient has been previously treated with one or more receptor tyrosine kinase inhibitors and has developed at least partial resistance to the one or more receptor tyrosine kinase inhibitors described herein.

In some embodiments, the selected chemotherapeutic agent is selected from the group consisting of entrectinib, NVP-TAE684, rebastinib, Compound 2, and any pharmaceutically acceptable salt thereof.

In some embodiments, the cancer is selected from anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, and papillary thyroid cancer, or any combination thereof. In some embodiments, the biological sample from the patient includes sputum, bronchoalveolar lavage, pleural effusion, tissue, whole blood, serum, plasma, buccal scrape, saliva, cerebrospinal fluid, urine, stool, circulating tumor cells, circulating nucleic acids, bone marrow, or any combination thereof.

In some embodiments, the knowledge of the presence of the one or more molecular alterations is acquired from an analytical assay selected from nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay, or any combination thereof. In some embodiments, the analytical assay is an electrophoretic mobility assay in which a nucleic acid sequence encoding the mutation is detected by amplifying the nucleic acid region corresponding to the mutation in the receptor tyrosine kinase gene and comparing the electrophoretic mobility of the amplified nucleic acid to the electrophoretic mobility of the corresponding region in a wild-type receptor tyrosine kinase gene. In some embodiments, the analytical assay is an allele-specific polymerase chain reaction or next-generation sequencing. In some embodiments, the analytical assay is a nucleic acid hybridization assay comprising contacting nucleic acids from the biological sample with a nucleic acid probe comprising a nucleic acid sequence complementary to a nucleic acid sequence encoding the one or more mutations and further including a detectable label.

In some embodiments, the knowledge of the presence of the one or more molecular alterations is acquired from an antibody-based assay selected from ELISA, immunohistochemistry, western blotting, mass spectrometry, flow cytometry, protein-microarray, immunofluorescence, and a multiplex detection assay, or any combination thereof. In some embodiments, the antibody-based assay includes one or more antibodies that specifically bind to one or more of TrkA, TrkB, TrkC, ALK, and ROS1 polypeptides.

In some embodiments, the selected chemotherapeutic agent, or a pharmaceutically acceptable salt thereof, is administered as a single therapeutic agent or in combination with one or more additional therapeutic agents.

In one aspect, some embodiments disclosed herein relate to methods for treating cancer in a patient, comprising (a) identifying a patient having one or more mutations at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) selecting a chemotherapeutic agent that is appropriate for treating said patient having said one or more mutations; and (c) administering a therapeutically effective amount of the selected chemotherapeutic agent to the patient.

In one aspect, some embodiments disclosed herein relate to methods for selecting a patient having cancer who is predicted to have an increased risk of unresponsiveness to treatment with a therapeutic regimen, comprising (a) acquiring knowledge of the presence of one or more mutations in a biological sample from said patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; and (b) selecting the patient as predicted to have an increased risk of unresponsiveness to treatment with a therapeutic regimen if one or more said mutations is detected in the biological sample, or selecting the patient as predicted to not have an increased risk of unresponsiveness to treatment with a therapeutic regimen if none of said one or more mutations is detected in the biological sample, wherein the therapeutic regimen includes administering to said selected patient a therapeutically effective amount of one or more chemotherapeutic agents. In some embodiments, the one or more chemotherapeutic agents is entrectinib, rebastinib, NVP-TAE684, staurosporine, or Compound 2, or a pharmaceutically acceptable salt thereof. In some embodiments, the methods further include treating the patient selected as having an increased risk of unresponsiveness to treatment with the therapeutic regimen. In some embodiments, the treating includes administering to the patient a therapeutic agent that is appropriate for treating a patient having one or more of the mutations. In some embodiments, the treating includes administering to said patient a therapeutic agent that is effective against multiple receptor tyrosine kinases.

In one aspect, some embodiments disclosed herein relate to methods for identifying a compound suitable for treatment of cancer in a patient who has become resistant to an inhibitor of a receptor tyrosine kinase resulting from one or more mutations in the receptor tyrosine kinase, comprising (a) acquiring knowledge of the presence of one or more mutations in a biological sample from said patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) determining the ability of the compound to inhibit the receptor tyrosine kinase having one or more of the mutations; and (c) identifying a compound as suitable for treatment of the patient if the compound inhibits the receptor tyrosine kinase having one or more of the mutations.

In one aspect, some embodiments disclosed herein relate to methods for selecting a treatment regimen for a patient having cancer, comprising (a) acquiring knowledge of the presence of one or more mutations in a biological sample from the patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; and (b) selecting an appropriate treatment regimen for the patient based on whether one or more of the mutations is present is the biological sample.

In one aspect, some embodiments disclosed herein relate to methods for predicting the outcome of a treatment regimen for a patient having cancer, comprising (a) acquiring knowledge of the presence of one or more mutations in a biological sample from the patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9, wherein the presence of one or more of the mutations in the biological sample is indicative of an increased unresponsiveness in the patient to the treatment regimen.

In one aspect, some embodiments disclosed herein relate to methods for treating a patient having a cancer tumor, comprising (a) determining the presence of a nucleic acid encoding a mutated Trk protein in a tumor sample from said patient, wherein said mutated Trk protein mutation comprises at least one mutation at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) selecting a Trk inhibitor appropriate for the treatment of said tumor; and (c) administering said Trk inhibitor to the patient.

In one aspect, some embodiments disclosed herein relate to methods for treating a patient having a cancer tumor, wherein the cancer tumor contains a mutated Trk gene, and wherein the mutated Trk gene within the cancer tumor shows resistance or acquired resistance to treatment with Trk inhibitors. The method includes administering a therapeutically effective amount of a Trk inhibitor that is active against a polypeptide encoded by the mutated Trk gene to a patient in need thereof, optionally in combination with radiotherapy, radio-immunotherapy and/or tumor resection by surgery.

In one aspect, some embodiments disclosed herein relate to methods for treating cancer in a patient comprising the steps of (a) selecting a patient with cancer having a Trk mutation; and (b) administering to the patient an inhibitor that is active against one or more of said Trk mutations.

In one aspect, some embodiments disclosed herein relate to methods for treating a patient having a cancer tumor, comprising (a) determining the presence of one or more mutations in the DNA sequence encoding a Trk protein in a tumor sample from the patient, the one or more mutations is at a position corresponding to an amino acid residue selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) selecting a Trk inhibitor appropriate for the treatment of the tumor; and (c) administering the Trk inhibitor to the patient.

In one aspect, some embodiments disclosed herein relate to methods for treating a cancer in a patient bearing a Trk mutation, wherein said subject has become resistant to at least one Trk inhibitor, comprising administering to said patient an effective amount of one or more inhibitors effective against multiple receptor tyrosine kinases.

The foregoing summary is illustrative only and is not intended to be in any way limiting. In addition to the illustrative aspects, alternatives, and features described above, further aspects, alternatives, objects and features of the disclosure will become fully apparent from the drawings and the following detailed description and the claims.

### BRIEF DESCRIPTION OF THE DRAWINGS

FIG. 1 is an alignment of the kinase domains from human receptor tyrosine kinases TrkA (NCBI Accession No. NP_002520.2; SEQ ID NO: 1), TrkB (NCBI Accession No. NP_006171.2; SEQ ID NO: 3), TrkC (NCBI Accession No. NP_001012338.1; SEQ ID NO: 5), ALK (NCBI Accession No. NM_004304.4; SEQ ID NO: 7), and ROS (NCBI Accession No. NP_002935.2; SEQ ID NO: 9). The sequence alignment of FIG. 1 was generated using the program CLUSTAL 2.1 with default settings. The amino acid numbering of each aligned sequence is with reference to the full-length polypeptide sequence indicated by the corresponding SEQ ID NO. In the alignment figure shown herein, a dash in an aligned sequence represents a gap, i.e., a lack of amino acid at that position. As discussed in detail below, several conserved amino acid residues and polypeptide motifs with high degree of conservation have been identified from this sequence comparison analysis. The amino acid residues corresponding to the kinase domain of each aligned sequence are indicated between parentheses. Asterisks identify identical and conserved amino acids among the aligned sequences. Boxed letters identify the amino acid residues within the aligned sequences that correspond to the conserved V573, F589, G595, and G667 residues of TrkA.

FIG. 2 is a brief description of some of the cell lines used in the experiments described at the Examples section.

FIG. 3 is a schematic illustration of a strategy for generating inhibitor-resistant cell lines and the subsequent characterization.

FIG. 4 illustrates an exemplary scheme for the selection of entrectinib-resistant KM12 cells.

FIG. 5 is a graphical summary of the results obtained from growth inhibition studies described in the Examples section herein where KM12 cells of Set A grown in media containing 0-30 nM entrectinib for 3 days upshifted.

FIG. 6 is a graphical summary of the results obtained from growth inhibition studies described in the Examples section herein where KM12 cell grown in media containing increasing concentrations of entrectinib for 4 weeks.

FIG. 7 is the sequencing results showing that the KM12 cell pools of Set B described at Example 4 were found to possess two point mutations at position G595 (G595R) and G667 (G677C) in the TrkA kinase domain.

FIG. 8 illustrates an exemplary scheme for the selection of entrectinib-resistant BaF3-tel/trkA cells.

FIG. 9 illustrates the establishment of BaF3-tel/trkA cell pools that developed resistance to 10 nM entrectinib after 2-week selection.

FIG. 10 is a graphical illustration of the reduced sensitivity of the KM12 cells of Set B to entrectinib, as described in detail at Example 4.

FIGS. 11A and 11B are graphical illustration of the results obtained from growth inhibition studies showing that the 10 nM entrectinib-resistant Baf3-trkA (A) cell pools displayed >100 fold higher IC50 compared to parental cells.

FIGS. 12A-12E show that withdrawing entrectinib from the 10 nM-resistant Baf3-trkA cell pools did not affect the resistance phenotype. Also shown in this figure is some exemplary inhibitory activity of RTK inhibitors in these cells.

FIG. 13 is a summary of the results from the 1^{st} RT-PCR and sequencing analysis of the kinase domain of TrkA, as described at Examples 4 and 5.

FIG. 14 shows that a G->A substitution in the TrkA kinase domain (Exon 14) in entrectinib-10 nM resistant-BaF3-tel/trkA cells. As a control, the Figure also shows that the TrkA sequence of the 100-nM entrectinib treated KM12 cell pools of Set A possessed wild-type sequence. The G->A single base substitution is indicated (encircled).

FIG. 15 is a summary of a sequence analysis experiment, which confirmed the presence of G595R mutation in entrectinib-resistant BaF3-tel/trkA-10nMA cells. The G->A single base substitution is indicated (encircled).

FIG. 16 shows a tridimensional modeling of the TrkA kinase domain illustrating that the G595 and G667C substitutions in the TrkA protein interferes with entrectinib binding to the ATP pocket of Trk polypeptide.

FIG. 17 shows a tridimensional modeling of the ALK kinase domain illustrating that the G1202 substitution interferes with entrectinib binding to the ATP pocket of ALK polypeptide, which is similar to the G595R and G2032R substitutions in TrkA and ROS1, respectively.

FIG. 18 is a summary of the results from the 2^{nd} RT-PCR and sequencing analysis of the kinase domain of TrkA in KM12 and BaF3-tel/TrkA cell lines, as described at Examples 4 and 5.

FIG. 19 is a summary of the results obtained from a sequence analysis experiment, which identified an additional G667C mutation in Exon 15 the in KM12 set B and BaF3-tel/trkA-12 nM entrectinib-resistant pools.

FIG. 20 is a sequencing chromatogram illustrating that DNA samples from KM12 cell pools show clean sequencing data for both G595R and G667C mutations, suggesting the pools derived from clonal cells. The G->T single base substitution is indicated (encircled).

FIG. 21 is a sequencing chromatogram illustrating that DNA samples from entrectinib-12 nM entrectinib-resistant BaF3-tel/trkA cells contain a mixture of G and T for G667C mutation.

FIG. 22 illustrates an exemplary scheme for subcloning of BaF3-tel/trkA-12nMA2 and 12nMB3 pools.

FIG. 23 is a summary of the results obtained from the sequencing analysis of twelve isolated clones derived from the subcloning experiment described at FIG. 22 above.

FIG. 24 illustrates an exemplary screening protocol and cells lines used in the experiments described in the Examples section.

FIG. 25 is a summary of the IC50 values of a number of chemical compounds that were tested against 7 cell lines including entrectinib-resistant BaF3-tel/trkA cells, as described in detail at Example 6.

FIG. 26 shows the biochemical IC50s of a list of candidate compounds against a number of kinases.

FIG. 27 illustrates another screening protocol and cells lines used in the experiments described in the Examples section.

FIG. 28 a summary of the IC50 values of a number of chemical compounds that were tested against entrectinib-resistant BaF3-Tel/TrkA cells, as described in detail at Example 6.

FIG. 29 is a general scheme of the study of the effect of entrectinib on RTKs and Proteins in the down-stream signal transduction pathway.

FIG. 30 is a summary of the results obtained from the characterization of entrectinib-resistant mutant cell line 10 nM BaF3-tel/trkA-containing G595R by using Western Blot analysis.

FIG. 31 is a summary of the results obtained from experiments comparing the phosphorylation level of TrkA and down-stream signal molecules in BaF3-Tel-TrkA and BaF3-Tel-TrkA-10nMA (G595R) cell lines by Western Blot analysis.

FIG. 32 illustrates a general scheme for the identification of point mutations as the primary resistance mechanism in entrectinib-resistant KM12 cell pools of Set B that is resistant to entrectinib.

FIG. 33 provides an overview of the cellular IC50 determination procedure described in further detail in Examples section.

FIG. 34 is a graphical illustration of the growth inhibition of BaF3-TPM3-TrkA cells and BaF3-TPM3-TrkA_G595R mutant cells by entrectinib.

FIG. 35 depicts an exemplary experimental design for Western Blot analysis of BaF3-fusion Trks cells treated with Entrectinib.

FIG. 36 is a summary of the results obtained from Western Blot analyses that were performed on BaF3-TPM3-TrkA cells and BaF3-TPM3-TrkA-G595R mutant cells.

FIG. 37 summarizes the results obtained from Western Blot analyses that were performed on BaF3-TPM3-TrkA cells and BaF3-TPM3-TrkA-G595R mutant cells.

### DETAILED DESCRIPTION OF THE DISCLOSURE

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be used, and other changes may be made, without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Some Definitions

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

“About” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof.

As used herein, anaplastic lymphoma kinase (ALK) refers to ALK tyrosine kinase receptor or CD246 (cluster of differentiation 246), for example a human enzyme encoded by the ALK gene and has the UniProt identified ALK_HUMAN.

As used herein, the term “antibody” refers to an immunoglobulin that specifically binds to, and is thereby defined as complementary with, a particular spatial and polar organization of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained.

The terms “monoclonal antibody,” “mAb” and “MAB” refer to an antibody that is an immunoglobulin produced by a single clone of lymphocytes which recognizes only a single epitope on an antigen. For example, a monoclonal antibody useful for the methods disclosed herein displays a single binding specificity and affinity for a particular epitope of one or more tyrosine kinases.

The term “polyclonal antibody” as used herein refers to a composition of different antibody molecules which is capable of binding to or reacting with several different specific antigenic determinants on the same or on different antigens. The variability in antigen specificity of a polyclonal antibody is located in the variable regions of the individual antibodies constituting the polyclonal antibody, in particular in the complementarity determining regions (CDRs). Preferably, the polyclonal antibody is prepared by immunization of an animal with the target tyrosine kinases or portions thereof. Alternatively, the polyclonal antibody may be prepared by mixing multiple monoclonal antibodies having desired specificity to a target tyrosine kinase.

The term “biological sample,” as used herein, encompasses a variety of sample types obtained from an organism. In some embodiments, a biological sample can be used in a diagnostic or monitoring assay. The biological sample may be obtained or derived from a healthy tissue, a diseased tissue or a tissue suspected of being diseased tissue. The biological sample may be a sample obtained from a biopsy taken, for example, during a surgical procedure. The biological sample may be collected via means of fine needle aspiration, scraping or washing a cavity to collects cells or tissue therefrom. The biological sample may be of a tumor such as, for example, solid and hematopoietic tumors as well as of neighboring healthy tissue. The biological sample may be a smear of individual cells or a tissue section. The term encompasses blood, blood components comprising plasma and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses clinical samples, and also includes cells in cell culture, cell supernatants, cell lysates, cell extracts, cell homogenates, subcellular components comprising synthesized proteins, serum, plasma, bodily and other biological fluids, and tissue samples. The biological sample can contain compounds that are not naturally intermixed with the cell or tissue in nature such as preservatives, anticoagulants, buffers, fixatives, nutrients, antibiotics or the like. In some embodiments, the sample biological is preserved as a frozen sample or as formaldehyde- or paraformaldehyde-fixed paraffin-embedded (FFPE) tissue preparation. For example, the biological sample can be embedded in a matrix, e.g., an FFPE block or a frozen sample.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

The term “chemotherapeutic agent” and “therapeutic agent”, which are used interchangeably herein, refers to a chemical substance, such as a cytotoxic or cytostatic agent, that is used to treat a condition, particularly cancer. In some embodiments, the chemotherapeutic agents include AZ-23, BMS-754807, bosutinib, cabozantinib, ceritinib, crizotinib, entrectinib, foretinib, GNF 5837, GW441756, imatinib mesylate, K252a, LOXO-101, MGCD516, nilotinib hydrochloride monohydrate, NVP-TAE684, PF-06463922, rebastinib, staurosporine, sorafenib tosylate, sunitinib malate, and TSR-011, and any pharmaceutically acceptable salts thereof.

As used herein the terms “combination” and “in combination with” mean the administration of a therapeutic agent described herein together with at least one additional pharmaceutical or medicinal agent (e.g., an anti-cancer agent), either sequentially or simultaneously. For example, the term encompasses dosing simultaneously, or within minutes or hours of each other, or on the same day, or on alternating days, or dosing the therapeutic agent described herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the therapeutic agent described herein is dosed.

As used herein, “contact” in reference to specificity or specific binding means two molecules are close enough so that short range non-covalent chemical interactions, such as Van der Waal forces, hydrogen bonding, hydrophobic interactions, and the like, dominate the interaction of the molecule.

The term “cell line” as used herein refers to one or more generations of cells which are derived from a clonal cell. The term “clone,” or “clonal cell,” refers to a single cell which is expanded to produce an isolated population of phenotypically similar cells (i.e. a “clonal cell population”).

As used herein, the term “expression” refers to the process of converting genetic information of a polynucleotide into RNA through transcription, which is typically catalyzed by an enzyme, RNA polymerase, and, where the RNA encodes a polypeptide, into protein, through translation of mRNA on ribosomes to produce the encoded protein.

The term “immunohistochemistry”, as used herein, refers to the process of localizing antigens (e.g. proteins) in biological samples, cells and/or cells of a tissue section exploiting the principle of antibodies binding specifically to antigens. Immunohistochemical staining is widely used in the diagnosis of abnormal cells such as those found in cancerous tumors. Specific molecular markers are characteristic of particular cellular events, such as cell proliferation or cell death. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore thus employing the principles of immunofluorescence. Immunohistochemistry can also be used to evaluate tumor content in the sample on which qPCR is carried out in order to account for the fact that qPCR result will be influenced by the amount of tumor tissue present.

As used herein, the term “one or more molecular alterations” means any variation in the genetic or protein sequence in or more cells of a patient as compared to the corresponding wild-type genes or proteins. One or more molecular alterations include, but are not limited to, genetic mutations, gene amplifications, splice variants, deletions, insertions/deletions, gene rearrangements, single-nucleotide variations (SNVs), insertions, and aberrant RNA/protein expression.

A “multiplexed assay,” as used herein, refers to an assay in which multiple assay reactions, e.g. simultaneous assays of multiple target biomarkers, are carried out in a single reaction chamber and/or and analyzed in a single separation and detection format. “Multiplex identification”, as used herein, refers to the simultaneous identification of one or more target biomarkers in a single mixture. For example, a two-plex assay refers to the simultaneous identification, in a single reaction mixture, of two different target biomarkers.

The terms “nucleic acid molecule” and “polynucleotide” are used interchangeably herein, and refer to RNA and DNA molecules or mixture or hybrid thereof. In some embodiments, nucleic acid molecules comprise cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

As used herein, “ROS1” refers to ROS1 receptor tyrosine-protein kinase, for example the ROS1 receptor tyrosine-protein kinase having the UniProt designation ROS1_HUMAN.

“Selectively binds” is used herein to refer to the situation in which one member of a specific intra- or inter-species binding pair will not show any significant binding to molecules other than its specific intra- or inter-species binding partner (e.g., an affinity of about 50-fold less or more preferably 100-fold less), which means that only minimal cross-reactivity occurs.

“Specific”, as used herein in reference to the binding of two molecules or a molecule and a complex of molecules, refers to the specific recognition of one for the other and the formation of a stable complex, as compared to substantially less recognition of other molecules and the lack of formation of stable complexes with such other molecules. Preferably, “specific,” in reference to binding, means that to the extent that a molecule forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

As used herein, the term “tropomyosin receptor kinase” refers to any members of the family of tropomyosin receptor kinases (Trks) that are activated by peptide hormones of the neurotrophin family. Examples of tropomyosin receptor kinase include, but are not limited to, TrkA, TrkB, and TrkC. As used herein, the term “TrkA” refers to the wild-type tropomyosin receptor kinase A having the UniProt identifier NTRK1_HUMAN. As used herein, the term “TrkB” refers to the wild-type tropomyosin receptor kinase B having the UniProt identifier NTRK2_HUMAN. As used herein, the term “TrkC” refers to the wild-type tropomyosin receptor kinase C having the UniProt identifier NTRK3_HUMAN. TrkA, TrkB and TrkC are also referred to by those of skill in the art as Trk1, Trk2 and Trk3, respectively. A reference to TrkA is a reference to Trk1. A reference to TrkB is a reference to Trk2. A reference to TrkC is a reference to Trk3.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

Receptor Tyrosine Kinases and Diseases Associated with their Activities

Neurotrophins control many aspects of neuronal survival and differentiation in the vertebrate nervous system by binding and signaling through the trk family of receptor tyrosine kinases (RTK). Gene families encoding RTKs with fundamental roles in nervous system have been shown to be highly conserved throughout evolution (Gad et al., J. Neurobiol. July; 60(1):12-20, 2004). Examples of the receptor tyrosine kinase include, but are not limited to, epidermal growth factor receptor family (EGFR), platelet-derived growth factor receptor (PDGFR) family, vascular endothelial growth factor receptor (VEGFR) family, nerve growth factor receptor (NGFR) family, fibroblast growth factor receptor family (FGFR) insulin receptor family, ephrin receptor family, Met family, and Ror family. Each family may comprise one or more family member that possesses characteristic structural and/or functional similarities.

Human Trk family proteins are receptor tyrosine kinases composed of three family members, TrkA, TrkB and TrkC. These proteins bind with high affinity to, and mediate the signal transduction induced by the neurotrophin family of ligands whose prototype members are Nerve Growth Factor (NGF), Brain-Derived Neurotrophic Factor (BDNF) and Neurotrophin 3-5 (NT 3-5). In addition, a co-receptor lacking enzymatic activity, p75, has been identified which binds all neurotrophins (NTs) with low affinity and regulates neurotrophin signaling. A critical role of the Trks and their ligands during the development of the central and peripheral nervous systems have been established through gene disruption studies in mice. In particular, TrkA-NGF interaction was shown as a requirement for the survival of certain peripheral neuron populations involved in mediating pain signaling. It has been shown that increased expression of TrkA also correlates with an increased level of pain in the case of pancreatic cancer (Zhu, et al., Journal of Clinical Oncology, 17:2419-2428 (1999)). Increased expression of NGF and TrkA was also observed in human osteoarthritis chondrocytes (Iannone et al., Rheumatology 41:1413-1418 (2002)).

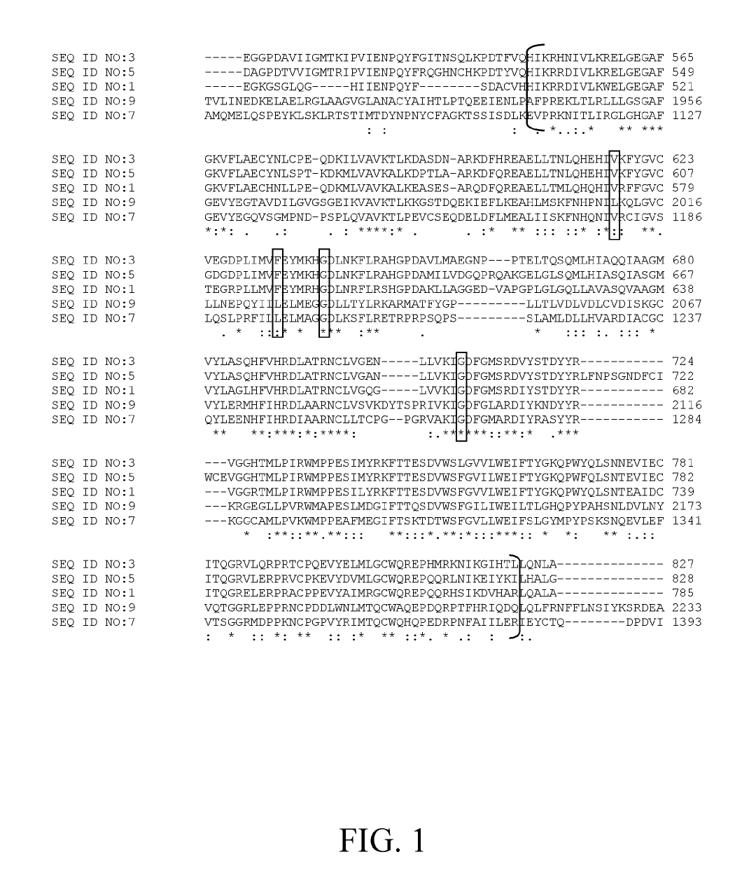

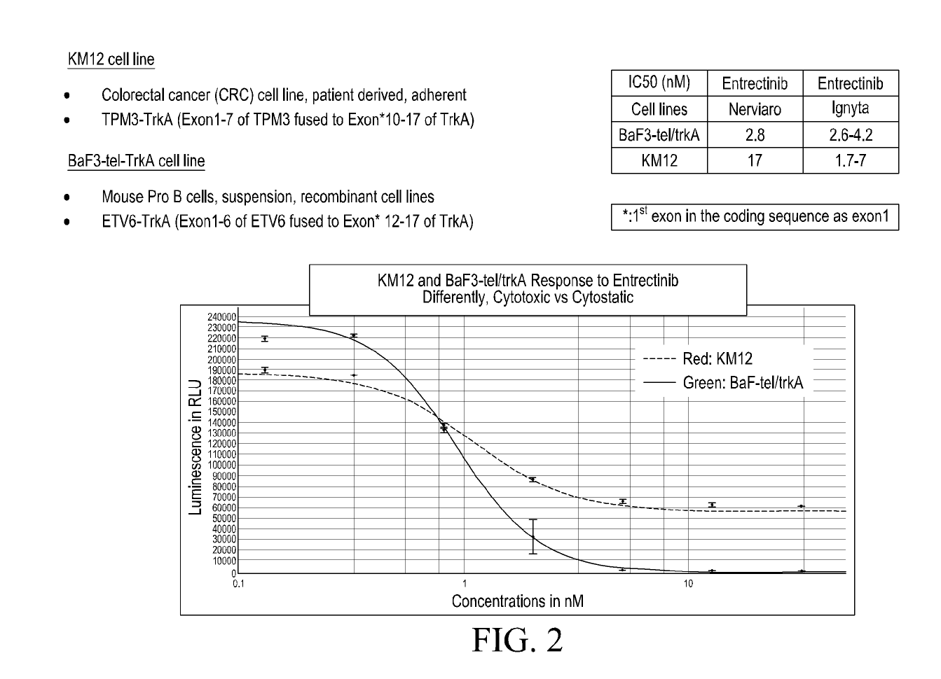

Although the amino acid sequences of various NTRK polypeptides differ in length, the relative positions of residues subject to the molecular alterations and mutations in accordance with the methods of the present invention are conserved (see, e.g., Gad et al., J. Neurobiol. July; 60(1):12-20, 2004; and TABLE 1 and FIG. 1). The molecular alterations and mutations described in the present disclosure in terms of amino acid positions correspond to the amino acid residue numbers of the human TrkA polypeptide (SEQ ID NO: 1). For examples, residue 639 of the human TrkB (disclosed herein as SEQ ID NO: 3) corresponds to residue 595 of the human TrkA polypeptide (SEQ ID NO: 1), which corresponds to residue 623 of the human TrkC polypeptide (SEQ ID NO: 5), residue 1202 of the human ALK polypeptide (SEQ ID NO: 7), and residue 2032 of the human ROS1 polypeptide (SEQ ID NO: 9). As another example, residue 709 of the human TrkB (disclosed herein as SEQ ID NO: 3) corresponds to residue 667 of the human TrkA polypeptide (SEQ ID NO: 1), which corresponds to residue 696 of the human TrkC polypeptide (SEQ ID NO: 5), residue 1269 of the human ALK polypeptide (SEQ ID NO: 7), and residue 2101 of the human ROS1 polypeptide (SEQ ID NO: 9). As yet another example, residue 619 of the human TrkB (disclosed herein as SEQ ID NO: 3) corresponds to residue 573 of the human TrkA polypeptide (SEQ ID NO: 1), which corresponds to residue 603 of the human TrkC polypeptide (SEQ ID NO: 5), residue 1182 of the human ALK polypeptide (SEQ ID NO: 7), and residue 2012 of the human ROS1 polypeptide (SEQ ID NO: 9). Non-limiting examples of conserved residues, motif, domains, and regions of correspondence relevant to the one or more molecular alterations in the TrkA polypeptide sequence disclosed herein are set forth in FIG. 1 and TABLE 1. Based on such correspondence, the corresponding conserved positions in the NRTK sequences not specifically disclosed herein can be readily determined by one of skill in the art.

TABLE 1
Concordant positions of exemplary conserved amino acid residues
in human TrkA, TrkB, TrkC, ALK and ROS1 polypeptides. Throughout
the present disclosure, the TrkA polypeptide is commonly used
as reference sequence in comparative sequence analysis because
structural features and residues important for the kinase
activity and physiological function of this polypeptide has
been most extensively characterized.

| TrkA | TrkB | TrkC | ALK | ROS1 |
|---|---|---|---|---|
| (SEQ ID | (SEQ ID | (SEQ ID | (SEQ ID | (SEQ ID |
| NO: 1) | NO: 3) | NO: 5) | NO: 7) | NO: 9) |
| V573 | V619 | V603 | V1182 | L2012 |
| F589 | F633 | F617 | L1196 | L2026 |
| E590 | E634 | E618 | E1197 | E2027 |
| M592 | M636 | M620 | M1199 | M2029 |
| G595 | G639 | G623 | G1202 | G2032 |
| D596 | D640 | D624 | D1203 | D2033 |
| L597 | L641 | L625 | L1204 | L2034 |
| K665 | K707 | K694 | K61267 | K2019 |
| I666 | I708 | I695 | I1268 | I2100 |
| G667 | G709 | G696 | G1269 | G2101 |
| D668 | D710 | D697 | D1270 | D2102 |
| F669 | F711 | F698 | F1271 | F2103 |
| G670 | G712 | G699 | G1272 | G2104 |

Accordingly, in some embodiments of the methods disclosed herein, the one or more mutations in a receptor tyrosine kinase polypeptide sequence include one or more amino acid deletions, insertions, or substitutions at one or more of the positions corresponding to conserved amino acid residues: V573, F589, E590, M592, G595, D596, L597, K665, 1666, G667, D668, F669, and G670, or combinations thereof, of the polypeptide of SEQ ID NO: 1. In some embodiments, the one or more mutations in a receptor tyrosine kinase polypeptide sequence can include one or more amino acid deletions, insertions, or substitutions at one or more of the positions corresponding to conserved amino acid residues: V619, F633, E634, M636, G639, D640, L641, K707, I708, G709, D710, F711, G712, and combinations of any thereof, of the polypeptide of SEQ ID NO: 3. In some embodiments, the one or more mutations in a receptor tyrosine kinase polypeptide sequence include one or more amino acid deletions, insertions, or substitutions at one or more of the positions corresponding to conserved amino acid residues: V603, F617, E618, M620, G623, D624, L625, K694, I695, G696, D697, F698, G699, and combinations of any thereof, of the polypeptide of SEQ ID NO: 5. In some embodiments, the one or more mutations in a receptor tyrosine kinase polypeptide sequence include one or more amino acid deletions, insertions, or substitutions at one or more of the positions corresponding to conserved amino acid residues: V1182, L1196, E1197, M1199, G1202, D1203, L1204, K61267, I1268, G1269, D1270, F1271, G1272, and combinations of any thereof, of the polypeptide of SEQ ID NO: 7. In some embodiments, the one or more mutations in a receptor tyrosine kinase polypeptide sequence include one or more amino acid deletions, insertions, or substitutions at one or more of the positions corresponding to conserved amino acid residues: L2012, L2026, E2027, M2029, G2032, D2033, L2034, K2019, I2100, G2101, D2102, F2103, G2104, and combinations of any thereof, of the polypeptide of SEQ ID NO: 9.

In some embodiments of the methods disclosed herein, the one or more mutations in a receptor tyrosine kinase polypeptide sequence include one or more amino acid deletions, insertions, or substitutions at one or more of the positions corresponding to conserved amino acid residues V573, F589, G595, G667, and combination thereof, of the polypeptide of SEQ ID NO: 1. In some embodiments, the one or more mutations in a receptor tyrosine kinase polypeptide sequence include one or more amino acid deletions, insertions, or substitutions at one or more of the positions corresponding to conserved amino acid residues V619, F633, G639, G709, and a combination thereof, of the polypeptide of ID NO: 3. In some embodiments, the one or more mutations in a receptor tyrosine kinase polypeptide sequence include one or more amino acid deletions, insertions, or substitutions at one or more of the positions corresponding to conserved amino acid residues V603, F617, G623, G696, and a combination thereof, of the polypeptide of SEQ ID NO: 5. In some embodiments, the one or more mutations in a receptor tyrosine kinase polypeptide sequence include one or more amino acid deletions, insertions, or substitutions at one or more of the positions corresponding to conserved amino acid residues V1182, L1196, G1202, G1269, and a combination thereof, of the polypeptide of SEQ ID NO: 7. In some embodiments, the one or more mutations in a receptor tyrosine kinase polypeptide sequence include one or more amino acid deletions, insertions, or substitutions at one or more of the positions corresponding to conserved amino acid residues L2012, L2026, G2032, G2101, and a combination thereof, of the polypeptide of SEQ ID NO: 9.

In some embodiments of the methods disclosed herein, the one or more mutations in a receptor tyrosine kinase polypeptide sequence include an amino acid deletion, insertion, or substitution at a position corresponding to the conserved amino acid residue V573 of the polypeptide of SEQ ID NO: 1. In some embodiments, the one or more mutations in a receptor tyrosine kinase polypeptide sequence include an amino acid deletion, insertion, or substitution at a position corresponding to a Val-to-Met substitution V573M of the polypeptide of SEQ ID NO: 1. In some embodiments of the methods disclosed herein, the one or more mutations in a receptor tyrosine kinase polypeptide sequence include an amino acid deletion, insertion, or substitution at a position corresponding to the conserved amino acid residue F589 of the polypeptide of SEQ ID NO: 1. In some embodiments, the one or more mutations in a receptor tyrosine kinase polypeptide sequence include an amino acid deletion, insertion, or substitution at a position corresponding to a Phe-to-Leu substitution F589L of the polypeptide of SEQ ID NO: 1. In some embodiments of the methods disclosed herein, the one or more mutations in a receptor tyrosine kinase polypeptide sequence include an amino acid deletion, insertion, or substitution at a position corresponding to the conserved amino acid residue G595 of the polypeptide of SEQ ID NO: 1. In some embodiments, the one or more mutations in a receptor tyrosine kinase polypeptide sequence include an amino acid deletion, insertion, or substitution at a position corresponding to a Gly-to-Arg substitution G595R of the polypeptide of SEQ ID NO: 1. In some embodiments, the one or more mutations in a receptor tyrosine kinase polypeptide sequence include an amino acid deletion, insertion, or substitution at a position corresponding to the conserved amino acid residue G667 of the polypeptide of SEQ ID NO: 1. In some embodiments, the one or more mutations in a receptor tyrosine kinase polypeptide sequence include an amino acid deletion, insertion, or substitution at a position corresponding to a Gly-to-Cys substitution G667C of the polypeptide of SEQ ID NO: 1. In some embodiments, the one or more mutations in a receptor tyrosine kinase polypeptide sequence include an amino acid deletion, insertion, or substitution at a position corresponding to a Gly-to-Ala substitution G667A of the polypeptide of SEQ ID NO: 1. In some embodiments, the one or more mutations in a receptor tyrosine kinase polypeptide sequence include an amino acid deletion, insertion, or substitution at a position corresponding to a Gly-to-Ser substitution G667S of the polypeptide of SEQ ID NO: 1.

In some embodiments of the methods disclosed herein, the one or more mutations in a receptor tyrosine kinase polypeptide sequence include an amino acid deletion, insertion, or substitution at a position corresponding to the conserved amino acid residue V619 of the polypeptide of SEQ ID NO: 3. In some embodiments, the one or more mutations in a receptor tyrosine kinase polypeptide sequence include an amino acid deletion, insertion, or substitution at a position corresponding to a Val-to-Met substitution V619M of the polypeptide of SEQ ID NO: 3. In some embodiments of the methods disclosed herein, the one or more mutations in a receptor tyrosine kinase polypeptide sequence include an amino acid deletion, insertion, or substitution at a position corresponding to the conserved amino acid residue F633 of the polypeptide of SEQ ID NO: 3. In some embodiments, the one or more mutations in a receptor tyrosine kinase polypeptide sequence include an amino acid deletion, insertion, or substitution at a position corresponding to a Phe-to-Leu substitution F633L of the polypeptide of SEQ ID NO: 3. In some embodiments of the methods disclosed herein, the one or more mutations in a receptor tyrosine kinase polypeptide sequence include an amino acid deletion, insertion, or substitution at a position corresponding to the conserved amino acid residue G639 of the polypeptide of SEQ ID NO: 3. In some embodiments, the one or more mutations in a receptor tyrosine kinase polypeptide sequence include an amino acid deletion, insertion, or substitution at a position corresponding to a Gly-to-Arg substitution G639R of the polypeptide of SEQ ID NO: 3. In some embodiments, the one or more mutations in a receptor tyrosine kinase polypeptide sequence include an amino acid deletion, insertion, or substitution at a position corresponding to the conserved amino acid residue G709 of the polypeptide of SEQ ID NO: 3. In some embodiments, the one or more mutations in a receptor tyrosine kinase polypeptide sequence include an amino acid deletion, insertion, or substitution at a position corresponding to a Gly-to-Cys substitution G709C of the polypeptide of SEQ ID NO: 3. In some embodiments, the one or more mutations in a receptor tyrosine kinase polypeptide sequence include an amino acid deletion, insertion, or substitution at a position corresponding to a Gly-to-Ala substitution G709A of the polypeptide of SEQ ID NO: 3. In some embodiments, the one or more mutations in a receptor tyrosine kinase polypeptide sequence include an amino acid deletion, insertion, or substitution at a position corresponding to a Gly-to-Ser substitution G709S of the polypeptide of SEQ ID NO: 3.

In some embodiments of the methods disclosed herein, the one or more mutations in a receptor tyrosine kinase polypeptide sequence include an amino acid deletion, insertion, or substitution at a position corresponding to the conserved amino acid residue V603 of the polypeptide of SEQ ID NO: 5. In some embodiments, the one or more mutations in a receptor tyrosine kinase polypeptide sequence include an amino acid deletion, insertion, or substitution at a position corresponding to a Val-to-Met substitution V603M of the polypeptide of SEQ ID NO: 5. In some embodiments of the methods disclosed herein, the one or more mutations in a receptor tyrosine kinase polypeptide sequence include an amino acid deletion, insertion, or substitution at a position corresponding to the conserved amino acid residue F617 of the polypeptide of SEQ ID NO: 5. In some embodiments, the one or more mutations in a receptor tyrosine kinase polypeptide sequence include an amino acid deletion, insertion, or substitution at a position corresponding to a Phe-to-Leu substitution F617L of the polypeptide of SEQ ID NO: 5. In some embodiments of the methods disclosed herein, the one or more mutations in a receptor tyrosine kinase polypeptide sequence include an amino acid deletion, insertion, or substitution at a position corresponding to the conserved amino acid residue G623 of the polypeptide of SEQ ID NO: 5. In some embodiments, the one or more mutations in a receptor tyrosine kinase polypeptide sequence include an amino acid deletion, insertion, or substitutions at a position corresponding to a Gly-to-Arg substitution G623R of the polypeptide of SEQ ID NO: 5. In some embodiments, the one or more mutations in a receptor tyrosine kinase polypeptide sequence include an amino acid deletion, insertion, or substitution at a position corresponding to the conserved amino acid residue G696 of the polypeptide of SEQ ID NO: 5. In some embodiments, the one or more mutations in a receptor tyrosine kinase polypeptide sequence include an amino acid deletion, insertion, or substitution at a position corresponding to a Gly-to-Cys substitution G696C of the polypeptide of SEQ ID NO: 5. In some embodiments, the one or more mutations in a receptor tyrosine kinase polypeptide sequence include an amino acid deletion, insertion, or substitution at a position corresponding to a Gly-to-Ala substitution G696A of the polypeptide of SEQ ID NO: 5. In some embodiments, the one or more mutations in a receptor tyrosine kinase polypeptide sequence include an amino acid deletion, insertion, or substitution at a position corresponding to a Gly-to-Ser substitution G696S of the polypeptide of SEQ ID NO: 5.

In some embodiments of the methods disclosed herein, the one or more mutations in a receptor tyrosine kinase polypeptide sequence include an amino acid deletion, insertion, or substitution at a position corresponding to the conserved amino acid residue V1182 of the polypeptide of SEQ ID NO: 7. In some embodiments, the one or more mutations in a receptor tyrosine kinase polypeptide sequence include an amino acid deletion, insertion, or substitution at a position corresponding to a Val-to-Met substitution V1182M of the polypeptide of SEQ ID NO: 7. In some embodiments of the methods disclosed herein, the one or more mutations in a receptor tyrosine kinase polypeptide sequence include an amino acid deletion, insertion, or substitution at a position corresponding to the conserved amino acid residue L1196 of the polypeptide of SEQ ID NO: 7. In some embodiments of the methods disclosed herein, the one or more mutations in a receptor tyrosine kinase polypeptide sequence include an amino acid deletion, insertion, or substitution at a position corresponding to the conserved amino acid residue G1202 of the polypeptide of SEQ ID NO: 7. In some embodiments, the one or more mutations in a receptor tyrosine kinase polypeptide sequence include an amino acid deletion, insertion, or substitution at a position corresponding to a Gly-to-Arg substitution G1202R of the polypeptide of SEQ ID NO: 7. In some embodiments, the one or more mutations in a receptor tyrosine kinase polypeptide sequence include an amino acid deletion, insertion, or substitution at a position corresponding to the conserved amino acid residue G1269 of the polypeptide of SEQ ID NO: 7. In some embodiments, the one or more mutations in a receptor tyrosine kinase polypeptide sequence include an amino acid deletion, insertion, or substitution at a position corresponding to a Gly-to-Cys substitution G1269C of the polypeptide of SEQ ID NO: 7. In some embodiments, the one or more mutations in a receptor tyrosine kinase polypeptide sequence include an amino acid deletion, insertion, or substitution at a position corresponding to a Gly-to-Ala substitution G1269A of the polypeptide of SEQ ID NO: 7. In some embodiments, the one or more mutations in a receptor tyrosine kinase polypeptide sequence include an amino acid deletion, insertion, or substitution at a position corresponding to a Gly-to-Ser substitution G1269S of the polypeptide of SEQ ID NO: 7.

In some embodiments of the methods disclosed herein, the one or more mutations in a receptor tyrosine kinase polypeptide sequence include an amino acid deletion, insertion, or substitution at a position corresponding to the conserved amino acid residue L2012 of the polypeptide of SEQ ID NO: 9. In some embodiments, the one or more mutations in a receptor tyrosine kinase polypeptide sequence include an amino acid deletion, insertion, or substitution at a position corresponding to a Leu-to-Met substitution L2012M of the polypeptide of SEQ ID NO: 9. In some embodiments of the methods disclosed herein, the one or more mutations in a receptor tyrosine kinase polypeptide sequence include an amino acid deletion, insertion, or substitution at a position corresponding to the conserved amino acid residue L2026 of the polypeptide of SEQ ID NO: 9. In some embodiments of the methods disclosed herein, the one or more mutations in a receptor tyrosine kinase polypeptide sequence include an amino acid deletion, insertion, or substitution at a position corresponding to the conserved amino acid residue G2032 of the polypeptide of SEQ ID NO: 9. In some embodiments, the one or more mutations in a receptor tyrosine kinase polypeptide sequence include an amino acid deletion, insertion, or substitution at a position corresponding to a Gly-to-Arg substitution G2032R of the polypeptide of SEQ ID NO: 9. In some embodiments, of the one or more mutations in a receptor tyrosine kinase polypeptide sequence include an amino acid deletion, insertion, or substitution at a position corresponding to the conserved amino acid residue G2101 of the polypeptide of SEQ ID NO: 9. In some embodiments, the one or more mutations in a receptor tyrosine kinase polypeptide sequence include an amino acid deletion, insertion, or substitution at a position corresponding to a Gly-to-Cys substitution G2101C of the polypeptide of SEQ ID NO: 9. In some embodiments, the one or more mutations in a receptor tyrosine kinase polypeptide sequence include an amino acid deletion, insertion, or substitution at a position corresponding to a Gly-to-Ala substitution G2101A of the polypeptide of SEQ ID NO: 9. In some embodiments, the one or more mutations in a receptor tyrosine kinase polypeptide sequence include an amino acid deletion, insertion, or substitution at a position corresponding to a Gly-to-Ser substitution G2101S of the polypeptide of SEQ ID NO: 9.

With respect to a nucleotide-based assay, degeneracy of the genetic code provides the possibility to substitute at least one base of the protein encoding sequence of a gene with a different base without affecting the amino acid sequence of the polypeptide produced from the mutated gene to be changed. Hence, the polynucleotide sequence of the probes, primers used in the methods disclosed herein may also have any base sequence that has been changed from any polynucleotide sequence described herein by substitution in accordance with degeneracy of the genetic code. References describing codon usage are readily available to one of ordinary skill in the art.

It is further contemplated that polynucleotide and polypeptide sequences of a receptor tyrosine kinase disclosed herein may be altered by various methods, and that these alterations may result in polynucleotide and polypeptide sequences having one or more mutations different than the sequences disclosed herein. As such, any of the polynucleotide and polypeptide sequences disclosed herein may be altered in various ways comprising amino acid substitutions, deletions, truncations, and insertions of one or more amino acids of the polypeptide sequences set forth in the Sequence Listing, comprising up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100, about 105, about 110, about 115, about 120, about 125, about 130 or more amino acid substitutions, deletions or insertions. Methods for such manipulations are generally known in the art.

Accordingly, other possible molecular alterations and mutations will be apparent to those skilled in the art based on the amino acid mutations in the kinase domain of the NRTK polypeptides that have been reported herein to confer resistance to one or more of the therapeutic agents described herein.

### Methods for Selecting/Treating Cancer Patient and Methods for Identifying Compounds Suitable for the Treatment of Cancer

In one aspect, the present disclosure provides methods for treating cancer in patient, comprising (a) acquiring knowledge of the presence of one or more molecular alterations in a biological sample from the patient, wherein the one or more molecular alterations includes one or more mutations in one or more receptor tyrosine kinase polypeptides, wherein one or more receptor tyrosine kinase polypeptides is selected from TrkA, TrkB, TrkC, ALK and ROS1; (b) selecting a chemotherapeutic agent appropriate for the treatment of the cancer; and (c) administering a therapeutically effective amount of the selected chemotherapeutic agent to the patient.

In another aspect, some embodiments disclosed herein relate to methods for selecting a treatment regimen for a patient having cancer, comprising (a) acquiring knowledge of the presence of one or more mutations in a biological sample from the patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; and (b) selecting an appropriate treatment regimen for the patient based on whether one or more of the mutations is present is the biological sample.

In yet another aspect, some embodiments disclosed herein relate to methods for predicting the outcome of a treatment regimen for a patient having cancer, comprising (a) acquiring knowledge of the presence of one or more mutations in a biological sample from the patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9, wherein the presence of one or more of the mutations in the biological sample is indicative of an increased unresponsiveness in the patient to the treatment regimen.

In another aspect, some embodiments disclosed herein relate to methods for treating a patient having a cancer tumor, comprising (a) determining the presence of a nucleic acid encoding a mutated Trk protein in a tumor sample from the patient, wherein the mutated Trk protein comprises at least one mutation at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) selecting a Trk inhibitor appropriate for the treatment of said tumor; and (c) administering said Trk inhibitor to the patient.

In one aspect, some embodiments disclosed herein relate to methods for treating a patient having a cancer tumor, wherein the cancer tumor contains a mutated Trk gene, and wherein the mutated Trk gene within the cancer tumor shows resistance or acquired resistance to treatment with Trk inhibitors. The methods, in some embodiments, include administering a therapeutically effective amount of a Trk inhibitor that is active against a polypeptide encoded by the mutated Trk gene to a patient in need thereof, optionally in combination with radiotherapy, radio-immunotherapy and/or tumor resection by surgery.

In one aspect, some embodiments disclosed herein relate to methods for treating cancer in a patient comprising the steps of (a) selecting a patient with cancer having a Trk mutation; and (b) administering to said patient an inhibitor that is active against one or more of said Trk mutations.

In one aspect, some embodiments disclosed herein relate to methods for treating a patient having a cancer tumor, comprising (a) determining the presence of a mutated Trk protein in a tumor sample from said patient, said mutated Trk protein comprises at least one mutation at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) selecting a Trk inhibitor appropriate for the treatment of the tumor; and (c) administering the Trk inhibitor to the patient.

In one aspect, some embodiments disclosed herein relate to methods for treating a cancer in a patient bearing a Trk mutation, wherein said subject has become resistant to at least one Trk inhibitor, comprising administering to said patient an effective amount of one or more inhibitors effective against multiple receptor tyrosine kinases.

In one aspect, some embodiments disclosed herein relate to methods for identifying a compound suitable for treatment of cancer in a patient who has become resistant to an inhibitor of a receptor tyrosine kinase resulting from one or more mutations in the receptor tyrosine kinase, comprising (a) acquiring knowledge of the presence of one or more mutations in a biological sample from said patient, wherein the one or more mutations is at an amino acid position selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9; (b) determining the ability of the compound to inhibit the receptor tyrosine kinase having one or more of the mutations; and (c) identifying a compound as suitable for treatment of the patient if the compound inhibits the receptor tyrosine kinase having one or more of the mutations.

Implementations of the methods according to one or more of the above aspects and other aspects of the disclosure can include one or more of the following features. In some embodiments, the one or more mutations described herein includes one or more amino acid substitutions in the kinase catalytic domain of the receptor tyrosine kinase polypeptide. In some embodiments, the one or more one amino acid substitutions is at a position corresponding to an amino acid residue selected from the group consisting of the amino acid residues identified in FIG. 1 and/or TABLE 1 as conserved residues, and combinations of any thereof. In some embodiments, the one or more amino acid substitutions is at a position corresponding to an amino acid residue selected from V573, F589, G595 and G667 of the TrkA polypeptide of SEQ ID NO:1; V619, F633, G639 and G709 of the TrkB polypeptide of SEQ ID NO:3; V603, F617, G623 and G696 of the TrkC polypeptide of SEQ ID NO:5; V1182, L1196, G1202 and 1269 of the ALK polypeptide of SEQ ID NO:7; and L2012, L2026, G2032 and 2101 of the ROS1 polypeptide of SEQ ID NO:9.

In some embodiments, the one or more amino acid substitutions is at a position corresponding to amino acid residue V573 of the TrkA polypeptide of SEQ ID NO: 1. In some embodiments, the one or more amino acid substitutions is a Val-to-Met substitution at a position corresponding to amino acid residue V573 of the TrkA polypeptide (V573M). In some embodiments, the one or more amino acid substitutions is at a position corresponding to amino acid residue F589 of the TrkA polypeptide of SEQ ID NO: 1. In some embodiments, the one or more amino acid substitutions is a Phe-to-Leu substitution at a position corresponding to amino acid residue F589 of the TrkA polypeptide (F589L). In some embodiments, the one or more amino acid substitutions is at a position corresponding to amino acid residue G595 of the TrkA polypeptide of SEQ ID NO: 1. In some embodiments, the one or more amino acid substitutions is a Gly-to-Arg substitution at a position corresponding to amino acid residue G595 of the TrkA polypeptide (G595R). In some embodiments, the one or more amino acid substitutions is at a position corresponding to amino acid residue G667 of the TrkA polypeptide of SEQ ID NO: 1. In some embodiments, the one or more amino acid substitutions is a Gly-to-Cys substitution at a position corresponding to amino acid residue G667 of the TrkA polypeptide (G667C). In some embodiments, the one or more amino acid substitutions is a Gly-to-Ala substitution at a position corresponding to amino acid residue G667 of the TrkA polypeptide (G667A). In some embodiments, the one or more amino acid substitutions is a Gly-to-Ser substitution at a position corresponding to amino acid residue G667 of the TrkA polypeptide (G667S).

In some embodiments, the one or more amino acid substitutions is at a position corresponding to amino acid residue V619 of the TrkB polypeptide of SEQ ID NO: 3. In some embodiments, the one or more amino acid substitutions is a Val-to-Met substitution at a position corresponding to amino acid residue V619 of the TrkB polypeptide (V619M). In some embodiments, the one or more amino acid substitutions is at a position corresponding to amino acid residue F633 of the TrkB polypeptide of SEQ ID NO: 3. In some embodiments, the one or more amino acid substitutions is a Phe-to-Leu substitution at a position corresponding to amino acid residue F633 of the TrkB polypeptide (F633L). In some embodiments, the one or more amino acid substitutions is at a position corresponding to amino acid residue G639 of the TrkB polypeptide of SEQ ID NO: 3. In some embodiments, the one or more amino acid substitutions is a Gly-to-Arg substitution at a position corresponding to amino acid residue G639 of the TrkB polypeptide (G639R). In some embodiments, the one or more amino acid substitutions is at a position corresponding to amino acid residue G709 of the TrkB polypeptide of SEQ ID NO: 3. In some embodiments, the one or more amino acid substitutions is a Gly-to-Cys substitution at a position corresponding to amino acid residue G709 of the TrkB polypeptide (G709C). In some embodiments, the one or more amino acid substitutions is a Gly-to-Ala substitution at a position corresponding to amino acid residue G709 of the TrkB polypeptide (G709A). In some embodiments, the one or more amino acid substitutions is a Gly-to-Ser substitution at a position corresponding to amino acid residue G709 of the TrkB polypeptide (G709S).

In some embodiments, the one or more amino acid substitutions is at a position corresponding to amino acid residue V603 of the TrkC polypeptide of SEQ ID NO: 5. In some embodiments, the one or more amino acid substitutions is a Val-to-Met substitution at a position corresponding to amino acid residue V603 of the TrkC polypeptide (V603M). In some embodiments, the one or more amino acid substitutions is at a position corresponding to amino acid residue F617 of the TrkC polypeptide of SEQ ID NO: 5. In some embodiments, the one or more amino acid substitutions is at a position corresponding to amino acid residue G623 of the TrkC polypeptide of SEQ ID NO: 5. In some embodiments, the one or more amino acid substitutions is a Phe-to-Leu substitution at a position corresponding to amino acid residue F623 of the TrkC polypeptide (F623L). In some embodiments, the one or more amino acid substitutions is a Gly-to-Arg substitution at a position corresponding to amino acid residue G623 of the TrkC polypeptide (G623R). In some embodiments, the one or more amino acid substitutions is at a position corresponding to amino acid residue G696 of the TrkC polypeptide of SEQ ID NO: 5. In some embodiments, the one or more amino acid substitutions is a Gly-to-Cys substitution at a position corresponding to amino acid residue G696 of the TrkC polypeptide (G696C). In some embodiments, the one or more amino acid substitutions is a Gly-to-Ala substitution at a position corresponding to amino acid residue G696 of the TrkC polypeptide (G696A). In some embodiments, the one or more amino acid substitutions is a Gly-to-Ser substitution at a position corresponding to amino acid residue G696 of the TrkC polypeptide (G696S).

In some embodiments, the methods disclosed herein relate to treat, reduce the symptoms of, ameliorate the symptoms of, delay the onset of, or otherwise pharmaceutically address a cancer condition in a patient that has been previously treated with one or more receptor tyrosine kinase inhibitors and has developed at least partial resistance to one or more such inhibitors.

In some embodiments, the methods disclosed herein relate to treat, reduce the symptoms of, ameliorate the symptoms of, delay the onset of, or otherwise pharmaceutically address a cancer condition in a patient that has been previously treated with one or more receptor tyrosine kinase inhibitors and has developed at least partial resistance to one or more such inhibitors. Non-limiting examples of such receptor tyrosine kinase inhibitors include AZ-23, BMS-754807, bosutinib, cabozantinib, ceritinib, crizotinib, entrectinib, foretinib, GNF 5837, GW441756, imatinib mesylate, K252a, LOXO-101, MGCD516, nilotinib hydrochloride monohydrate, NVP-TAE684, PF-06463922, rebastinib, staurosporine, sorafenib tosylate, sunitinib malate, TSR-011, and any combinations thereof (TABLE 2). In some embodiments, the methods disclosed herein relate to treat, reduce the symptoms of, ameliorate the symptoms of, delay the onset of, or otherwise pharmaceutically address a cancer condition in a patient that has been previously treated with entrectinib.

TABLE 2
Non-limiting examples of chemotherapeutic agents

| Compound Name | CAS Registry No. | Chemical Name | Reference |
|---|---|---|---|
| crizotinib | 877399-52-5 | (R)-3-[1-(2,6-Dichloro-3- | U.S. Pat. No. |
| | | fluorophenyl)ethoxy]-5-[1-(piperidin-4- | 7,230,098 |
| | | yl)-1H-pyrazol-4-yl]pyridin-2-amine | |
| entrectinib | 1108743-60-7 | N-[5-(3,5-difluorobenzyl)-1H-indazol-3- | U.S. Pat. No. |
| | | yl]-4-(4-methyl-piperazin-1-yl)-2- | 8,299,057 |
| | | (tetrahydro-pyran-4-ylamino)-benzamide | |
| NVP-TAE684 | 761439-42-3 | 5-chloro-N2-[2-methoxy-4-[4-(4-methyl- | U.S. Pat. No. |
| | | 1-piperazinyl)-1-piperidinyl]phenyl]-N4- | 7,964,592 |
| | | [2-[(1-methylethyl)sulfonyl]phenyl]-2,4- | |
| | | Pyrimidinediamine | |
| foretinib | 937176-80-2 | l-N′-[3-fluoro-4-[6-methoxy-7-(3- | U.S. Pat. No. |
| | | morpholin-4-ylpropoxy)quinolin-4- | 8,497,284 |
| | | yl]oxyphenyl]-1-N-(4- | |
| | | fluorophenyl)cyclopropane-1,1- | |
| | | dicarboxamide | |
| BMS-754807 | 1001350-96-4 | (2S)-1-[4-[(5-cyclopropyl-1H-pyrazol-3- | U.S. Pat. No. |
| | | yl)amino]pyrrolo[2,1-f][1,2,4]triazin-2- | 7,534,792 |
| | | yl]-N-(6-fluoropyridin-3-yl)-2- | |
| | | methylpyrrolidine-2-carboxamide | |
| GNF 5837 | 1033769-28-6 | 1-[2-fluoro-5-(trifluoromethyl)phenyl]-3- | WO 2008073480 |
| | | [4-methyl-3-[[(3Z)-2-oxo-3-(1H-pyrrol-2- | |
| | | ylmethylidene)-1H-indol-6- | |
| | | yl]amino]phenyl]urea | |
| rebastinib | 1020172-07-9 | 4-[4-[(5-tert-butyl-2-quinolin-6-ylpyrazol- | U.S. Pat. No. |
| | | 3-yl)carbamoylamino]-3-fluorophenoxy]- | 7,790,756 |
| | | N-methylpyridine-2-carboxamide | |
| GW441756 | 504433-23-2 | 3-[(1-methylindol-3-yl)methylidene]-1H- | U.S. Pat. No. |
| | | pyrrolo[3,2-b]pyridin-2-one | 7,015,231 |
| cabozantinib | 849217-68-1 | 1-N-[4-(6,7-dimethoxyquinolin-4- | U.S. Pat. No. |
| | | yl)oxyphenyl]-1-N'-(4- | 7,579,473 |
| | | fluorophenyl)cyclopropane-1,1- | |
| | | dicarboxamide | |
| bosutinib | 380843-75-4 | 4-(2,4-dichloro-5-methoxyanilino)-6- | WO 2004075898 |
| | | methoxy-7-[3-(4-methylpiperazin-1- | |
| | | yl)propoxy]quinoline-3-carbonitrile | |
| Compound 2 | 1034974-86-1 | N-[5-(3,5-difluoro-benzenesulfonyl)-1H- | U.S. Pat. No. |
| | | indazol-3-yl]-2-((R)-2-methoxy-1-methyl- | 8,114,865 |
| | | ethylamino)-4-(4-methyl-piperazin-1-yl) | |
| | | benzamide | |
| TSR-011 | 1388225-79-3 | N-[1,3-dihydro-6-[[4-(1-hydroxy-1- | Journal of |
| | | methylethyl)-1-piperidinyl]methyl]-1-[cis- | Medicinal |
| | | 4-[[(1- | Chemistry, |
| | | methylethyl)amino]carbonyl]cyclohexyl]- | Volume 55, |
| | | 2H-benzimidazol-2-ylidene]-3,5-difluoro-, | Issue 14, |
| | | [N(E)]-benzamide, | pp. 6523-6540, |
| | | | 2012 |
| MGCD516 | 1123837-84-2 | N-[3-fluoro-4-[[2-[5-[[(2- | U.S. Pat. No. |
| | | methoxyethyl)amino]methyl]-2- | 8,404,846 |
| | | pyridinyl]thieno[3,2-b]pyridin-7- | |
| | | yl]oxy]phenyl]-N′-(4-fluorophenyl)-1,1- | |
| | | cyclopropanedicarboxamide | |
| ceritinib | 1032900-25-6 | 5-chloro-2-N-(5-methyl-4-piperidin-4-yl- | U.S. Pat. No. |
| | | 2-propan-2-yloxyphenyl)-4-N-(2-propan- | 8,372,858 |
| | | 2-ylsulfonylphenyl)pyrimidine-2,4- | |
| | | diamine | |
| LOXO-101 | 1223403-58-4 | (3S)-N-[5-[(2R)-2-(2,5-difluorophenyl)-1- | U.S. Pat. No. |
| | | pyrrolidinyl]pyrazolo[1,5-a]pyrimidin-3- | 8,513,263 |
| | | yl]-3-hydroxy-1-pyrrolidinecarboxamide | |
| PF-06463922 | 1454846-35-5 | (10R)-7-amino-12-fluoro-10,15,16,17- | U.S. Pat. No. |
| | | tetrahydro-2,10,16-trimethyl-15-oxo-2H- | 8,680,111 |
| | | 4,8-Methenopyrazolo[4,3- | |
| | | h][2,5,11]benzoxadiazacyclotetradecine- | |
| | | 3-carbonitrile | |
| AZ-23 | 915720-21-7 | 5-chloro-2-N-[(1S)-1-(5-fluoropyridin-2- | U.S. Pat. No. |
| | | yl)ethyl]-4-N-(3-propan-2-yloxy-1H- | 8,114,989 |
| | | pyrazol-5-yl)pyrimidine-2,4-diamine | |
| K252a | 99533-80-9 | 9,12-Epoxy-1H-diindolo[1,2,3-fg:3′,2′,1′- | U.S. Pat. No. |
| | | kl]pyrrolo[3,4-i][1,6]benzodiazocine-10- | 4,555,402 |
| | | carboxylic acid, 2,3,9,10,11,12- | |
| | | hexahydro-10-hydroxy-9-methyl-1-oxo-, | |
| | | methyl ester, (9S,10R,12R)- | |
| Staurosporine | 62996-74-1 | 9,13-Epoxy-1H,9H-diindolo[1,2,3- | Commercially |
| | | gh:3′,2′,1′-lm]pyrrolo[3,4- | available; |
| | | j][1,7]benzodiazonin-1-one, | Journal of |
| | | 2,3,10,11,12,13-hexahydro-10-methoxy- | Antibiotics |
| | | 9-methyl-11-(methylamino)-, | Volume 30, |
| | | (9S,10R,11R,13R)- | Issue 4, pp. |
| | | | 75-82, 1977 |

In some embodiments of the methods disclosed herein, the one or more mutations in a receptor tyrosine kinase polypeptide confers resistance or acquired resistance to treatment with entrectinib, rebastinib, or a pharmaceutically acceptable salt thereof.

Some embodiments of the methods disclosed herein comprise selecting a chemotherapeutic agent appropriate for the treatment of the cancer, and administering a therapeutically effective amount of the selected chemotherapeutic agent to the patient. Non-limiting examples of such chemotherapeutic agents include those listed in TABLE 2, or any pharmaceutically acceptable salt thereof. In some embodiments, the selected chemotherapeutic agent is selected from the group consisting of entrectinib, NVP-TAE684, rebastinib, Compound 2, and any pharmaceutically acceptable salt thereof.

The methods and compounds according to the present disclosure can be deployed for selecting and/or treating a patient having any cancer. Non-limiting examples of suitable cancers to be treated include anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, papillary thyroid cancer, or any combination thereof.

Some embodiments of the methods disclosed herein relate to treat, reduce the symptoms of, ameliorate the symptoms of, delay the onset of, or otherwise pharmaceutically address a cancer condition selected from anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, papillary thyroid cancer, in which one or more mutations in a receptor tyrosine kinase polypeptide selected from TrkA, TrkB, TrkC, ALK and ROS1 might play a role by selecting chemotherapeutic agent appropriate for the treatment of the cancer condition, and administering a therapeutically effective amount of the selected chemotherapeutic agent to the patient.

The types of biological samples suitable for use in the methods described herein are not particularly limited. In some embodiments, the biological sample comprises sputum, bronchoalveolar lavage, pleural effusion, tissue, whole blood, serum, plasma, buccal scrape, saliva, cerebrospinal fluid, urine, stool, circulating tumor cells, circulating nucleic acids, bone marrow, or any combination thereof. In yet some embodiments, the biological sample includes whole blood and blood components. In some embodiments, the blood component comprises plasma. In yet other embodiments, the biological sample includes cells or tissue. In some embodiments, the tissue is a tumor or cancer tissue.

In some embodiments of the methods disclosed herein, the acquiring knowledge of one or more molecular alterations from an analytical assay performed on a biological sample obtained from a patient. The analytical assay can generally be any analytical assay known to those having ordinary skill in the art, and can be for example an antibody-based assay, a nucleotide-based assay, or an enzymatic activity assay. Non-limited examples of suitable analytical assays include nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay. Other examples of suitable analytical assays include ELISA, immunohistochemistry, western blotting, mass spectrometry, flow cytometry, protein-microarray, immunofluorescence, a multiplex detection assay, or any combination thereof.

In some embodiments, an electrophoretic mobility assay is used to acquire the knowledge of the one or more molecular alterations in the biological sample obtained from a patient. For example, a nucleic acid sequence encoding the mutation is detected by amplifying the nucleic acid region corresponding to the one or more alterations in a receptor tyrosine kinase gene and comparing the electrophoretic mobility of the amplified nucleic acid to the electrophoretic mobility of the corresponding region in a wild-type receptor tyrosine kinase gene.

In some embodiments, the analytical assay used to acquire the knowledge of the one or more molecular alterations in the biological sample involves polymerase chain reactions (PCR) or nucleic acid amplification-based assays. A number of PCR-based analytical assays known in the art are suitable for the methods disclosed herein, comprising but not limited to real-time PCR, quantitative reverse transcription PCR (qRT-PCR), and PCR-RFLP assay.

In some embodiments, the analytical assay used to acquire the knowledge of the one or more molecular alterations in the biological sample involves determining a nucleic acid sequence and/or an amino acid sequence comprising the one or more molecular alterations. In some embodiments, the nucleic acid sequence comprising the one or more molecular alterations from a cancer patient is sequenced. In some embodiments, the sequence is determined by a next generation sequencing procedure. As used herein “next-generation sequencing” refers to oligonucleotide sequencing technologies that have the capacity to sequence oligonucleotides at speeds above those possible with conventional sequencing methods (e.g. Sanger sequencing), due to performing and reading out thousands to millions of sequencing reactions in parallel. Non-limiting examples of next-generation sequencing methods/platforms include Massively Parallel Signature Sequencing (Lynx Therapeutics); solid-phase, reversible dye-terminator sequencing (Solexa/Illumina); DNA nanoball sequencing (Complete Genomics); SOLiD technology (Applied Biosystems); 454 pyro-sequencing (454 Life Sciences/Roche Diagnostics); ion semiconductor sequencing (ION Torrent); and technologies available from Pacific Biosciences, Intelligen Bio-systems, Oxford Nanopore Technologies, and Helicos Biosciences.

Accordingly, in some embodiments, the NGS procedure used in the methods disclosed herein can comprise pyrosequencing, sequencing by synthesis, sequencing by ligation, or a combination of any thereof. In some embodiments, the NGS procedure is performed by an NGS platform selected from Illumina, Ion Torrent, Qiagen, Invitrogen, Applied Biosystem, Helicos, Oxford Nanopore, Pacific Biosciences, and Complete Genomics.

In some embodiments, FISH analysis can be used to identify the chromosomal mutations resulting in the one or more molecular alterations such as the mutated genes or mutated gene products (i.e. polypeptides) as described herein. For example, to perform FISH, at least a first probe tagged with a first detectable label can be designed to target a mutated gene of a mutated polypeptide, and at least a second probe tagged with a second detectable label can be designed to target the corresponding wild-type gene or wild-type polypeptide such that one of ordinary skill in the art observing the probes can determine that a relevant gene or gene product is present in the sample. Generally, FISH assays are performed using formalin-fixed, paraffin-embedded tissue sections that are placed on slides. For example, the DNA from the biological samples is denatured to single-stranded form and subsequently allowed to hybridize with the appropriate DNA probes that can be designed and prepared using methods and techniques known to those having ordinary skill in the art. Following hybridization, any unbound probe may be removed by a series of washes and the nuclei of the cells are counter-stained with DAPI (4′,6 diamidino-2-phenylindole), a DNA-specific stain that fluoresces blue. Hybridization of the probe or probes are viewed using a fluorescence microscope equipped with appropriate excitation and emission filters, allowing visualization of the fluorescent signals. Other variations of the FISH method known in the art are also suitable for evaluating a patient selected in accordance with the methods disclosed herein.

In some embodiments, the analytical assay used to acquire the knowledge of the one or more molecular alterations in the biological sample involves a nucleic acid hybridization assay. The term “hybridization”, as used herein, refers generally to the ability of nucleic acid molecules to join via complementary base strand pairing. Such hybridization may occur when nucleic acid molecules are contacted under appropriate conditions and/or circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, nucleic acid molecules are said to exhibit “complete complementarity” when every nucleotide of one of the molecules is complementary to its base pairing partner nucleotide of the other. Two molecules are said to be “minimally complementary” if they can hybridize to one another with sufficient stability to permit them to remain annealed to one another under at least conventional “low-stringency” conditions. In some instances, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions are said to be “hybridizable cognates” of the other nucleic acid molecules. Conventional stringency conditions are described by Sambrook et al., Molecular Cloning, A Laboratory Handbook, Cold Spring Harbor Laboratory Press, 1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment thereof to serve as a primer or probe it needs only be sufficiently complementary in sequence to be able to form a stable double-stranded structure under the particular solvent and salt concentrations employed.

Appropriate stringency conditions which promote DNA hybridization include, for example, 6.0× sodium chloride/sodium citrate (SSC) at about 45° C., followed by a wash of 2.0×SSC at about 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. These conditions are known to those skilled in the art, or can be found in Current Protocols in Molecular Biology, John Wiley & Sons, N.Y. (1989), 6.3.1-6.3.6. For example, low stringency conditions may be used to select nucleic acid sequences with lower sequence identities to a target nucleic acid sequence. One may wish to employ conditions such as about 0.15 M to about 0.9 M sodium chloride, at temperatures ranging from about 20° C. to about 55° C. High stringency conditions may be used to select for nucleic acid sequences with higher degrees of identity to the disclosed nucleic acid sequences (Sambrook et al., 1989, supra). In one embodiment, high stringency conditions involve nucleic acid hybridization in about 2×SSC to about 10×SSC (diluted from a 20×SSC stock solution containing 3 M sodium chloride and 0.3 M sodium citrate, pH 7.0 in distilled water), about 2.5× to about 5×Denhardt's solution (diluted from a 50× stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 50° C. to about 70° C. for several hours to overnight. High stringency conditions are preferably provided by 6×SSC, 5×Denhardt's solution, 100 mg/mL sheared and denatured salmon sperm DNA, and 0.1% (w/v) SDS, with incubation at 55 λC for several hours. Hybridization is generally followed by several wash steps. The wash compositions generally comprise 0.5×SSC to about 10×SSC, and 0.01% (w/v) to about 0.5% (w/v) SDS with a 15-min incubation at about 20° C. to about 70° C. Preferably, the nucleic acid segments remain hybridized after washing at least one time in 0.1×SSC at 65° C. In some instances, very high stringency conditions may be used to select for nucleic acid sequences with much higher degrees of identity to the disclosed nucleic acid sequences. Very high stringency conditions are defined as prehybridization and hybridization at 42° C. in 5×SSPE, 0.3% SDS, 200 μg/mL sheared and denatured salmon sperm DNA, and 50% formamide and washing three times each for 15 minutes using 2×SSC, 0.2% SDS at 70° C.

In some embodiments, the analytical assay used to acquire the knowledge of the one or more molecular alterations in the biological sample involves a nucleic acid hybridization assay that includes contacting nucleic acids derived from the biological sample with a nucleic acid probe comprising (1) a nucleic acid sequence complementary to a nucleic acid sequence encoding the one or more mutations and further comprising (2) a detectable label.

In some embodiments are provided such methods, wherein the knowledge of the presence of the one or more molecular alterations is obtained from an assay performed simultaneously on a plurality of biological samples. In some embodiments, the plurality of biological samples may be assayed in a multitest platform.

As used herein, the term “multitest platform” is intended to encompass any suitable means to contain one or more reaction mixtures, suspensions, or detection reactions. As such, the outcomes of a number of screening events can be assembled onto one surface, resulting in a “multitest platform” having, or consisting of multiple elements or parts to do more than one experiment simultaneously. It is intended that the term “multitest platform” encompasses protein chips, microtiter plates, multi-well plates, microcards, test tubes, petri plates, trays, slides, and the like. In some embodiments, multiplexing can further include simultaneously conducting a plurality of screening events in each of a plurality of separate biological samples. For example, the number of biological samples analyzed can be based on the number of spots on a slide and the number of tests conducted in each spot. In another example, the number of biological samples analyzed can be based on the number of wells in a multi-well plate and the number of tests conducted in each well. For example, 6-well, 12-well, 24-well, 48-well, 96-well, 384-well, 1536-well or 3456-well microtiter plates can be useful in the presently disclosed methods, although it will be appreciated by those in the art, not each microtiter well need contain a patient biological sample. Depending on the size of the microtiter plate and the number of the individual biological samples in each well, very high numbers of tests can be run simultaneously. In some embodiments, the plurality of biological samples includes at least 6, 12, 24, 48, 96, 200, 384, 400, 500, 1000, 1250, 1500, or 3000 sample.

In some embodiments, knowledge is acquired from an antibody-based assay, comprising but not limited to ELISA, immunohistochemistry, western blotting, mass spectrometry, flow cytometry, protein-microarray, immunofluorescence, a multiplex detection assay, or any combination thereof. In some embodiments, the antibody-based assay includes the use of one or more antibodies that selectively bind to one or more of TrkA, TrkB, TrkC, ALK, and ROS1 polypeptides.

In some embodiments of the methods disclosed herein, the chemotherapeutic agents, or a pharmaceutically acceptable salt thereof, are selected for administration or are administered to an individual or patient having cancer, optionally in combination with at least one additional chemotherapeutic agent. In some embodiments, entrectinib, rebastinib, NVP-TAE684, staurosporine, Compound 2, or a pharmaceutically acceptable salt thereof is used in the methods disclosed herein as the chemotherapeutic agent that is appropriate for treating cancer.

In some embodiments, the chemotherapeutic agents described herein, or a pharmaceutically acceptable salt thereof, is administered at a therapeutically effective amount to the patient. As used herein, the term “therapeutically effective amount” means that amount of the compound or compounds being administered which will relieve to some extent one or more of the symptoms of the disorder being treated. In reference to the treatment of a cancer, a therapeutically effective amount refers to that amount which has the effect of (1) reducing the size of a cancer tumor, (2) inhibiting (that is, slowing to some extent, preferably stopping) cancer tumor metastasis, (3) inhibiting to some extent (that is, slowing to some extent, preferably stopping) cancer tumor growth, and/or, (4) relieving to some extent (or, preferably, eliminating) one or more symptoms associated with the cancer.

This amount will vary depending upon a variety of factors, comprising but not limited to the characteristics of the bioactive compositions and formulations disclosed herein (comprising activity, pharmacokinetics, pharmacodynamics, and bioavailability thereof), the physiological condition of the subject treated (comprising age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the one or more bioactive compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more bioactive compositions and formulations disclosed herein and adjusting the dosage accordingly. Additional guidance with regard to this aspect can be found in, for example, Remington: The Science and Practice of Pharmacy, 21st Edition, Univ. of Sciences in Philadelphia (USIP), Lippincott Williams & Wilkins, Philadelphia, Pa., 2005.

In some embodiments of the methods disclosed herein, the selected chemotherapeutic agents, or a pharmaceutically acceptable salt thereof, is administered as a single therapeutic agent or in combination with one or more additional therapeutic agents.

In some embodiments of the methods disclosed herein, the selected chemotherapeutic agent, or a pharmaceutically acceptable salt thereof, are administered to a patient having or suffering from cancer in an amount ranging from about 200 mg/m² to about 1600 mg/m², or from about 200 mg/m² to about 1200 mg/m², or from about 200 mg/m² to about 1000 mg/m², or from about 400 mg/m² to about 1200 mg/m², or from about 400 mg/m² to about 1000 mg/m², or from about 800 mg/m² to about 1000 mg/m², or from about 800 mg/m² to about 1200 mg/m², or from about 800 mg/m² to about 1200 mg/m², or from about 800 mg/m² to about 1600 mg/m². In some embodiments, the chemotherapeutic agents described above are administered to the patient in an amount of about 200 mg/m², about 300 mg/m², about 400 mg/m², about 500 mg/m², about 600 mg/m², about 700 mg/m², about 800 mg/m², about 900 mg/m², about 1000 mg/m², about 1100 mg/m², about 1200 mg/m², about 1300 mg/m², about 1400 mg/m², about 1500 mg/m², about 1600 mg/m², about 1700 mg/m², about 1800 mg/m², about 1900 mg/m², or about 2000 mg/m². In some embodiments, the selected chemotherapeutic agent, or a pharmaceutically accepted salt thereof, is administered to a patient or individual having or suffering from cancer in multiple dosages for a treatment period of 2 to 50 days. In some embodiments, the selected chemotherapeutic agent, or a pharmaceutically accepted salt thereof, is administered to a patient or individual having or suffering from cancer in multiple dosages of about 50 to about 200 mg/kg per dose over a treatment period of 5 to 42 days. In some embodiments, the selected chemotherapeutic agent, or a pharmaceutically accepted salt thereof, is administered to a patient having or suffering from cancer with an oral dosage of about 60 mg/kg twice a day (BID), seven times per week. In some embodiments, the selected chemotherapeutic agent, or a pharmaceutically accepted salt thereof, is administered to a patient having or suffering from cancer with an oral dosage of about 60 mg/kg twice a day (BID), seven times per week for six weeks, on alternate weekly basis (i.e. one week on one week off).

Some embodiments include any of the methods described herein, the selected chemotherapeutic agent, or a pharmaceutically acceptable salt thereof, are administered to a patient having or suffering from cancer in an amount ranging from about 0.01 mg/kg to about 100 mg/kg, or from about 0.02 mg/kg to about 50 mg/kg, or from about 0.05 mg/kg to about 25 mg/kg, or from about 0.1 mg/kg to about 20 mg/kg, or from about 0.2 mg/kg to about 10 mg/kg, or from about 0.5 mg/kg to about 5 mg/kg, or from about 1 mg/kg to about 2 mg/kg.

In some embodiments of the methods disclosed herein, the chemotherapeutic agents described herein may be administered to a cancer patient in need thereof by administration to the patient of a pharmaceutical composition comprising one or more such agents. In particular, such pharmaceutical compositions may comprise one or more of the chemotherapeutic agents described herein, or a pharmaceutically acceptable salt thereof, and at least one pharmaceutically acceptable excipient.

In some embodiments, such pharmaceutical compositions can comprise a physical admixture of the various ingredients in solid, liquid, or gelcap form. Other embodiments can comprise at least two separated ingredients in a single dosage unit or dosage form, such as, for example, a two- or three-layer tablet in which at least two active ingredients are located in separate layers or regions of the tablet, optionally separated by a third material, such as, for example, a sugar layer or other inert barrier to prevent contact between the first two ingredients. In other embodiments, two or more active ingredients are separately formulated into individual dosage units, which are then packaged together for ease of administration. One embodiment comprises a package containing a plurality of individual dosage units. This embodiment may, for example, comprise a blister package. In one embodiment of a blister package, multiple blister-packed dosage units are present on a single sheet, and those units that are to be administered together are packaged in the same or adjacent blisters of the blister pack. Alternatively, any other packaging can be used in which two active ingredients are packaged together for concurrent or sequential use.

Some embodiments relate to the use of any of the chemotherapeutic agents as described herein, or a pharmaceutically acceptable salt thereof, in the manufacture of a medicament for the treatment of abnormal cell growth in a mammal. The present disclosure further relates to the use of any of the chemotherapeutic agents as described herein, or a pharmaceutically acceptable salt thereof, in the manufacture of a medicament for the treatment of abnormal cell growth in a mammal wherein the abnormal cell growth is cancerous or non-cancerous. In some embodiments, the abnormal cell growth is cancerous. In another embodiment, the abnormal cell growth is non-cancerous.

Some embodiments relate to pharmaceutical compositions comprising a chemotherapeutic agent described herein, or a pharmaceutically acceptable salt thereof, a pharmaceutically acceptable carrier and, optionally, at least one additional medicinal or pharmaceutical agent. In some embodiments, the at least one additional medicinal or pharmaceutical agent is an anti-cancer agent as described below.

The pharmaceutically acceptable carrier may comprise a conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

In some embodiments, the composition comprises a therapeutically effective amount of a compound as disclosed herein and a pharmaceutically acceptable carrier.

The compounds described herein may be formulated into pharmaceutical compositions as described below in any pharmaceutical form recognizable to the skilled artisan as being suitable. Pharmaceutical compositions of the disclosure comprise a therapeutically effective amount of at least one compound disclosed herein and an inert, pharmaceutically acceptable carrier or diluent.

To treat or prevent diseases or conditions mediated by one or more of the mutated receptor tyrosine kinase disclosed herein, a pharmaceutical composition is administered in a suitable formulation prepared by combining a therapeutically effective amount of at least one compound (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, comprising lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Further, the pharmaceutical composition may be incorporated into a skin patch for delivery of the drug directly onto the skin.

It will be appreciated that the actual dosages of the agents of this disclosure will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations may contain a compound or compounds, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg. Furthermore, the pharmaceutically acceptable formulations may contain a compound, or a salt or solvate thereof, in an amount of about 50 mg, about 100 mg, about 150 mg, about 200 mg, about 250 mg, about 300 mg, about 350 mg, about 400 mg, about 450 mg, or about 500 mg.

Additionally, the pharmaceutically acceptable formulations may contain a compound, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds disclosed herein, or salts or solvates thereof, may be administered to a mammal suffering from abnormal cell growth, such as a human, either alone or as part of a pharmaceutically acceptable formulation, once a week, once a day, twice a day, three times a day, or four times a day, or even more frequently.

Those of ordinary skill in the art will understand that with respect to the compounds, the particular pharmaceutical formulation, the dosage, and the number of doses given per day to a mammal requiring such treatment, are all choices within the knowledge of one of ordinary skill in the art and can be determined without undue experimentation.

Administration of the compounds disclosed herein may be effected by any method that enables delivery of the compounds to the site of action. These methods include oral routes, intraduodenal routes, parenteral injection (comprising intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10. 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in patients.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional alternatives are disclosed in further detail in the following examples, which are not in any way intended to limit the scope of this disclosure or the claims.

### Example 1

### Generation of KM12 and Ba/F3-Tel/TrkA Cell Lines Resistant to Entrectinib

This Example describes the generation of entrectinib-resistant KM12 cell lines and entrectinib-resistant BA/F3-TEL/TRKA cell lines.

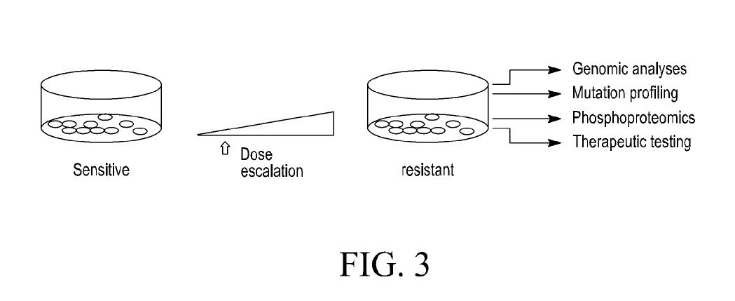

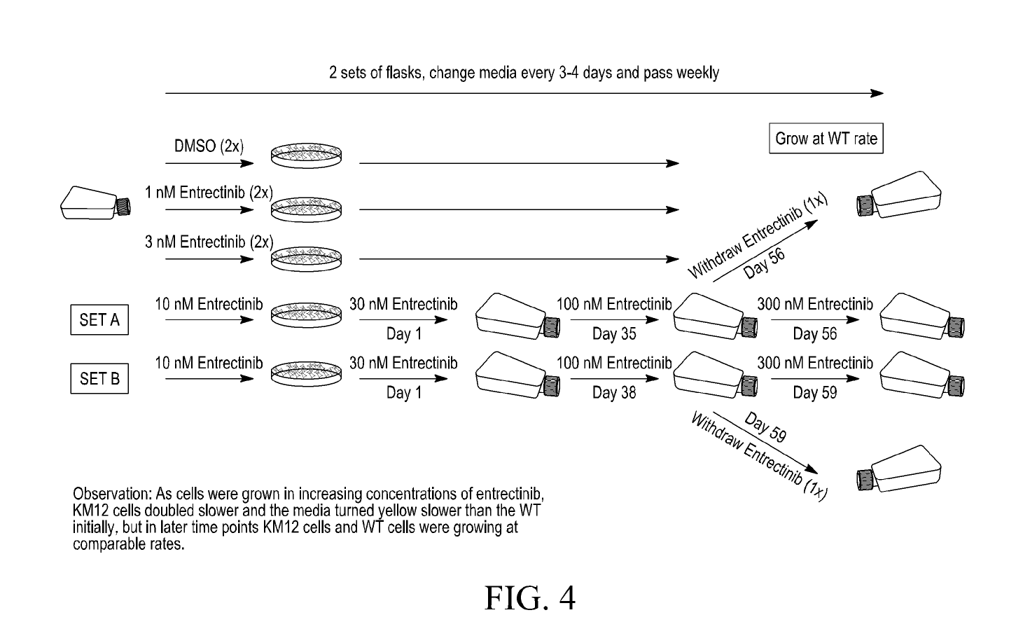

Schematic illustrations for selection and characterization of entrectinib-resistant KM12 cells are shown in FIG. 3 and FIG. 4. Cells of human colorectal cell line KM12 which harbors a TrkA fusion gene TPM3-TrkA were treated with 0, 1, 3, 10 nM of entrectinib initially in two independent sets of flasks (labeled A and B) in the complete culture media (RPMI medium (GIBCO®)+10% FBS (fetal bovine serum)+Penicillin and Streptomycin). Culture media containing 0.1% DMSO (i.e. untreated control) or entrectinib were changed every 3-4 days and the cultured cells were split about once a week. KM12 cells treated with 10 nM of entrectinib were subsequently cultured in the presence of 30 nM of entrectinib for approximately two weeks after initial treatment of 10 nM of entrectinib. Approximately four weeks after treatment, the cells were sequentially treated with 100 nM of entrectinib and, after another about 4-week period, were treated with 300 nM of entrectinib. At the end of each stage of treatment, cell aliquots were analyzed for growth inhibition by entrectinib for 3-day treatment using CellTiter Glo® (Promega). RNA/DNA was extracted from each of the cell samples. RT-PCR and sequencing analysis were performed by BioSettia (San Diego, Calif.).

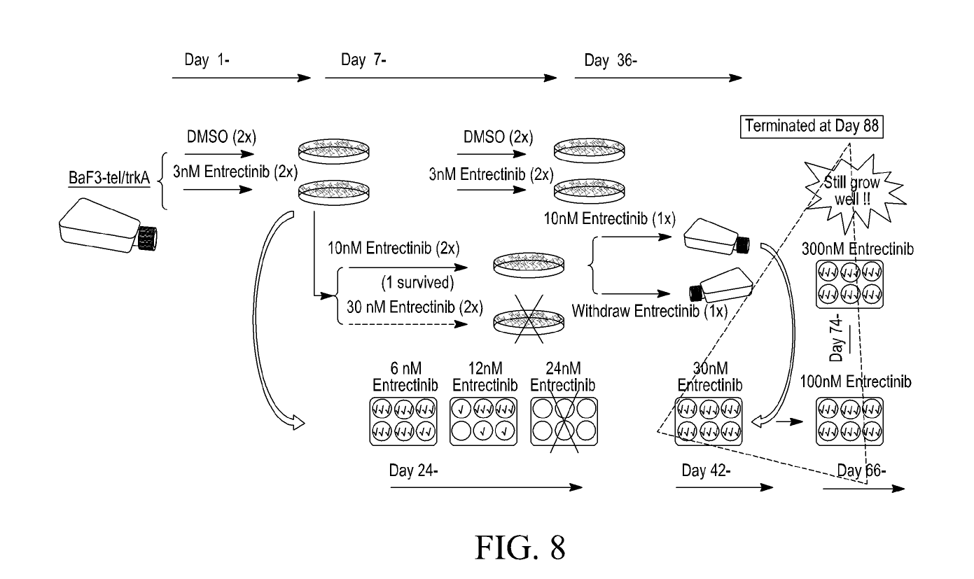

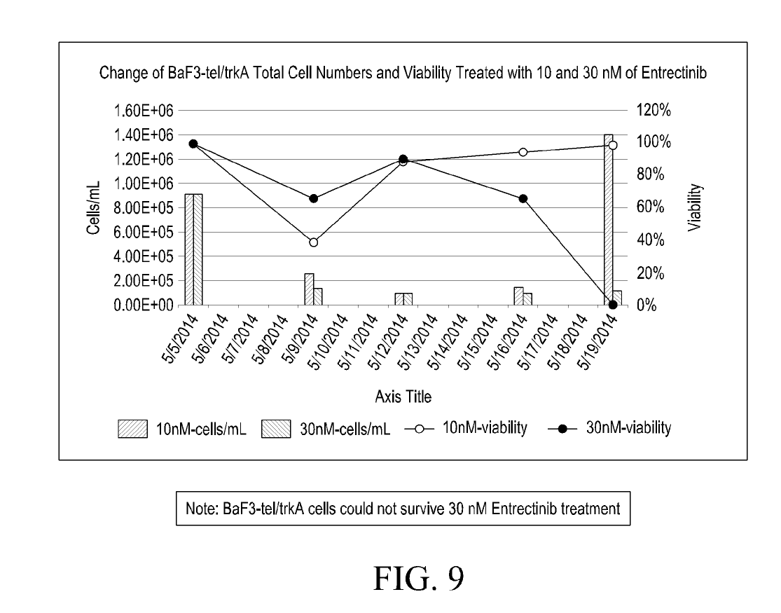

Schematic illustrations for selection and characterization of entrectinib-resistant Ba/F3-Tel/TrkA cells are shown in FIG. 3 and FIG. 8. Input Ba/F3-Tel/TrkA cell line was an engineered cell line harboring a recombinant TrkA fusion gene ETV6-TrkA. Ba/F3-Tel/TrkA cells were treated with 0 and 3 nM of entrectinib initially in two independent sets of flasks (labeled A and B) in the complete culture media (RPMI+10% FBS+Penicillin and Streptomycin). Seven days after the initial treatment, Ba/F3-Tel/TrkA cells treated with 3 nM of entrectinib were subsequently cultured in 10 and 30 nM of entrectinib in two independent sets (A and B) of flasks for approximately two weeks. Cell viability was evaluated by trypan blue and counted every two days (FIG. 9). At Day 24, the cells cultured at 3 nM of entrectinib were set up in triplicates (that is, 3 replicates from set A and 3 replicates from set B) and incubated with 6, 12, or 24 nM of entrectinib. The survived cell pools, named Ba/F3-Tel/TrkA-10nMA, Ba/F3-Tel/TrkA-6nMA1, Ba/F3-Tel/TrkA-6nMA2, Ba/F3-Tel/TrkA-6nMA3, Ba/F3-Tel/TrkA-6nMB1, Ba/F3-Tel/TrkA-6nMB2, Ba/F3-Tel/TrkA-6nMB3, Ba/F3-Tel/TrkA-12nMA1, Ba/F3-Tel/TrkA-12nMA2, Ba/F3-Tel/TrkA-12nMA3, Ba/F3-Tel/TrkA-12nMB2 and Ba/F3-Tel/TrkA-12nMB3, were expended and further characterized. Cells of the parental line and the entrectinib-resistant cells were analyzed for growth inhibition by entrectinib for 3-day treatment using CellTiter Glo (Promega). RNA/DNA was extracted from each of the cell samples. RT-PCR and sequencing analysis were performed by BioSettia (San Diego, Calif.).

### Example 2

### Cellular IC50 of RTK Inhibitors as Determined by Growth Inhibition Studies in KM12 Cells and BA/F3-TEL/TRKA Cells

This Example describes a general procedure developed to evaluate the anti-proliferative activity of RTK inhibitors, e.g. entrectinib, in parental KM12 cells and entrectinib-resistant KM12 cells. Cells of a parental KM12 line and an entrectinib-resistant KM12 line were trypsinized and seeded at 5,000 cells per well in 96-well assay white plates (Costar #3610), followed by an overnight incubation in the complete media without entrectinib. The next day, different concentrations of each of the RTK inhibitors, e.g. entrectinib (0 to 1 μM), were added to the wells. Each treatment condition was performed in duplicate. Similarly, Ba/F3-Tel/TrkA cells were seeded at 5,000 cells per well in 96-well assay white plates (Costar #3610) in the complete media without entrectinib and, on the next day, were treated with different concentrations of each of the RTK inhibitors, e.g. entrectinib (0 to 1 μM) in duplicates. Three days after incubation, cell viabilities were measured by luciferase-based ATP level detection using CellTiter-Glo® reagents (Promega) and IC50s were determined by 4-parameter curve fit with variable slope.

### Example 3

### Generation of Ba/F3-TPM3 and Ba/F3-TPM3-TrkA-G959R Cell Lines

This Example describes studies performed to generate transgenic Ba/F3 cells expressing either a wild-type protein TPM3-TrkA or a TPM3-TrkA-G595R fusion protein. A cDNA encoding TPM3-TrkA fusion was cloned from a KM12 parental cell line and entrectinib-resistant cells by a PCR-based technique and subsequently inserted into a lentiviral vector pVL-EF1a-MCS-IRES-Puro (BioSettia, San Diego, Calif.). After confirmation of the cDNA inserts by direct sequencing, vesicular stomatitis virus GP (VSVG)-pseudo-typed lentiviruses containing either the TPM3-TrkA cDNA or the TPM3-TrkA-G595R cDNA were transduced into the murine IL-3 dependent pro-B cell Ba/F3 at different multiplicity of infections (MOIs) with 8 μg/mL of polybrene (EMD Millipore). The transduced Ba/F3 cells were selected in the murine IL-3 containing RPMI media supplemented with 10% FBS and 1 μg/mL of puromycin for two weeks. The stable cell pools were further selected in RPMI media (GIBCO®) supplemented with 10% FBS (fetal bovine serum) and without murine IL-3 for 4 weeks.

### Example 4

### Isolation and Characterization of Entrectinib-Resistant KM12 Cells

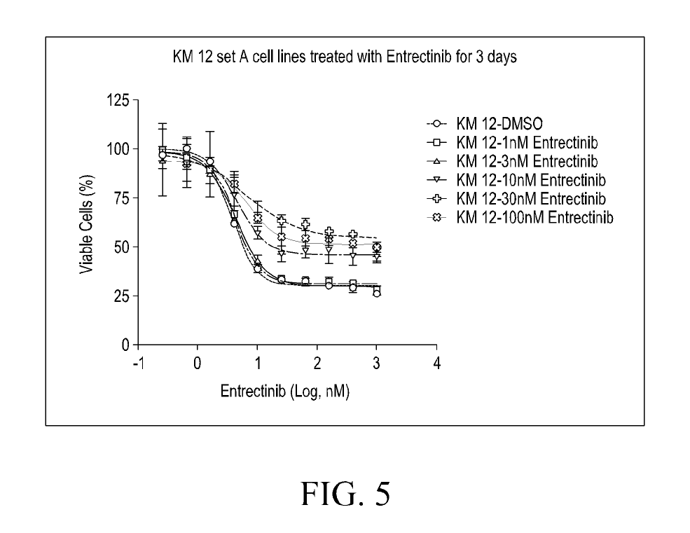

Six samples (duplicated samples at each treatment) of parental KM12 cells were treated with 0.01% DMSO (v/v), 1 nM entrectinib, 3 nM entrectinib, or 10 nM entrectinib for about two weeks. No apparent change in morphology and doubling time for the cells was observed. At the end of the two-week treatment, the duplicated samples of the KM12 cells treated with 10 nM entrectinib were cultured in growth media containing 30 nM of entrectinib. A slightly slower growth rate for these KM12-10 nM treated cells was observed. As shown in at FIG. 5, in a 3-day growth inhibition study, KM12 cells (Set A) cultured in DMSO (vehicle) and 1-10 nM entrectinib displayed overlapping growth inhibition curves and similar IC50 values (TABLE 3 and FIG. 5). However, KM12-30 nM-A treated cells displayed an upshifted growth curve and a ˜2-fold increase of IC50 value (TABLE 3), indicating a reduced sensitivity to entrectinib.

TABLE 3
IC50 values of the kinase inhibitor entrectinib in parental
KM12 cells and entrectinib-resistant cell lines

| | KM12- | KM12-1 | KM12-3 | KM12-10 | KM12-30 |
|---|---|---|---|---|---|
| Cell lines | DMSO-A | nM A | nM A | nM A | nM A |
| IC50 (nM) | 1.26 | 1.57 | 1.64 | 1.71 | 3.41 |
| R square | 0.9795 | 0.9841 | 0.9747 | 0.9615 | 0.9626 |

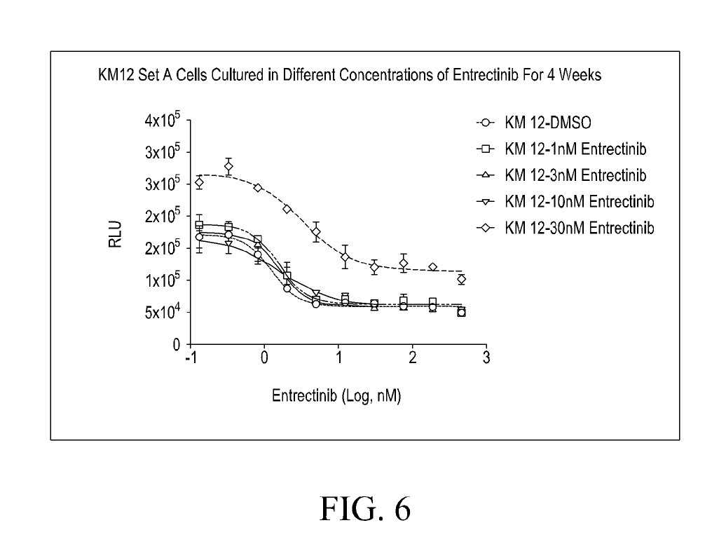

When entrectinib concentration in the culture media was increased from 30 to 100 μM for about 4 weeks, KM12 cells of Set A became even less sensitive to entrectinib, as indicated by the upshift of the bottom plateau (FIG. 6) and increased IC50 values (TABLE 4).

TABLE 4
IC50 values of the RTK inhibitor entrectinib in parental KM12 cells and
entrectinib-resistant cell lines

| | KM12- | | | | | |
|---|---|---|---|---|---|---|
| | DMSO | KM12-1 nM A | KM12-3 nM A | KM12-10 nM A | KM12-30 nM A | KM12-100 nM A |
| IC50 (nM) | 3.95 | 4.19 | 4.36 | 4.65 | 6.47 | 6.62 |
| R square | 0.9934 | 0.9952 | 0.9894 | 0.9343 | 0.9277 | 0.9055 |

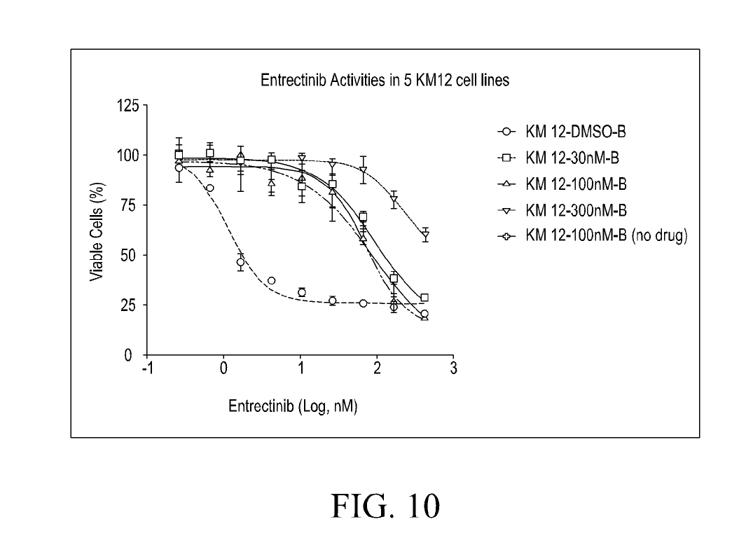

KM12 cells of Set B were also tested for their sensitivity to entrectinib (FIG. 10 and Table 5). As shown in TABLE 5, drastic increases of IC50 values in cells of Set B were observed when these cells were cultured at 30 nM and higher concentrations of entrectinib. Additionally, the change in cells cultured in media containing 100 nM of entrectinib for 4 weeks was found to be genetically stable. This conclusion was drawn from the observation that following a 4-weeks culture period in the presence of 100 nM of entrectinib (KM12-100nM-B), the IC50 values were found stable even after entrectinib was withdrawn from the cell culture media (KM12-100nM-B (no drug)), suggesting the change in these cells were at the genomic level.

TABLE 5
Sensitivity of KM12 cells of Set B to entrectinib
as determined by IC50 values

| | | | | | KM12- |
|---|---|---|---|---|---|
| | | KM12- | KM12- | KM12- | 100 |
| | KM12- | 30 | 100 | 300 | nM-B |
| entrectinib | DMSO-B | nM-B | nM-B | nM-B | (no drug) |
| IC50 (nM) | 1.14 | 93.83 | 70.37 | 208.7 | 85.41 |
| R square | 0.9790 | 0.9656 | 0.9624 | 0.9282 | 0.9567 |

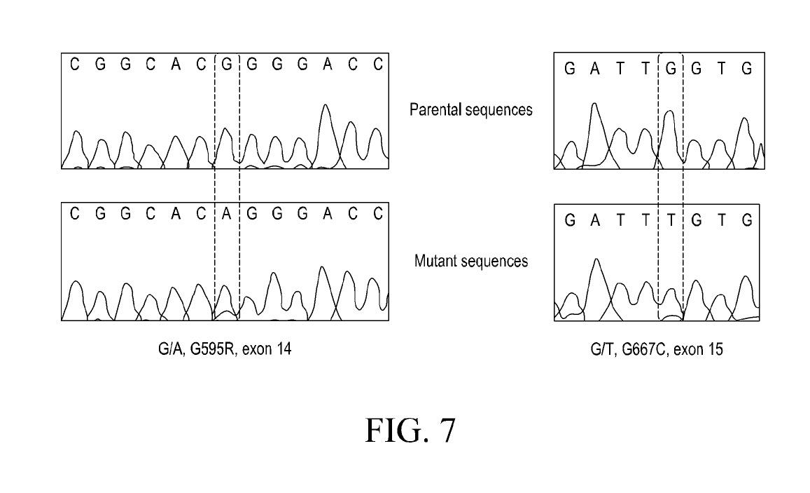

Following four weeks of treatment with 300 nM of entrectinib, RNA was isolated from each of the KM12 cell pools of both Set A and Set B, and subsequently subjected to RT-PCR and sequencing analysis. As shown at FIG. 7 and TABLE 6, no mutations in TrkA kinase domain were found in the cell pools of Set A, while the cells of Set B were found to possess two point mutations at position G595 and G667 in the TrkA kinase domain (TABLE 6). In particular, a Gly-to-Arg substitution was identified at residue G595 (i.e., G595R), and a Gly-to-Cys substitution was identified at residue G667 (i.e., G667C) (FIG. 7).

TABLE 6
Results from sequence analysis from entrectinib-
treated cell pools of Set A and Set B

| Sample ID | Mutations in TrkA Kinase Domain |
|---|---|
| KM12-DMSO-A | Wild-type sequences |
| KM12-30 nM-A | Wild-type sequences |
| KM12-100 nM-A | Wild-type sequences |
| KM12-100 nM-A (no drug) | Wild-type sequences |
| KM12-300 nM-A | Wild-type sequences |
| KM12-DMSO-B | Wild-type sequences |
| KM12-30 nM-B | G/T, Glycine/Cysteine, 667, exon 15 |
| KM12-100 nM-B | G/T, Glycine/Cysteine, 667, exon 15 |
| KM12-100 nM-B (no drug) | G/T, Glycine/Cysteine, 667, exon 15 |
| KM12-300 nM-B | G/A, Glycine/Arginine, 595, exon 14 |

Without being bound by any particular theory, two mechanisms of resistance are believed possible. In Set A, the resistance of KM12 could be a bypass mechanism, in which other signal transduction pathways have been affected. This possibility is supported by the observation that there is no mutation in the TPM3-trkA gene. In Set B, the change of nucleotide G to T (see, TABLE 6 and FIG. 7) resulted in a missense mutation (G667C) in exon 15 in KM12 cells cultured in entrectinib between 30-100 nM. However, when the cells were cultured in entrectinib concentrations ranging from 100 nM to 300 nM for another 4 weeks, a G to A change (FIG. 7) resulted in G595R in exon 14, but no G667C mutation was observed in these cells.

KM12 cells (cultured in 100 nM entrectinib) bearing the G667C mutation were found to be genetically stable because withdrawing the 100 nM of entrectinib for 4 weeks did not reverse the G667C mutation (TABLE 6). In the sequence alignment of FIG. 1, the amino acid numbering of TrkA is with reference to the full-length sequence of TrkA having GenBank accession number NP_002520.2. The corresponding amino acid numberings of TrkB and TrkC are shown in TABLES 1 and 7.

TABLE 7
Concordance positions of conserved amino acid residues in the
kinase domains of human TrkA, TrkB, and TrkC polypeptides

| | | | Length | | |
|---|---|---|---|---|---|
| Gene Name | Species | GeneBank ID | (aa) | residue | Residue |
| NTRK1, | Human | NP_002520.2 | 796 | G595 | G667 |
| TrkA | | NM_002529.3 | | | |
| NTRK2, | Human | NP_006171.2 | 838 | G639 | G709 |
| TrkB | | NM_006180.3 | | | |
| NTRK3, | Human | NP_001012338.1 | 839 | G623 | G696 |
| TrkC | | NM_001012338.2 | | | |

### Example 5

### Isolation and Characterization of Entrectinib-Resistant Ba/F3-Tel/trkA Cells

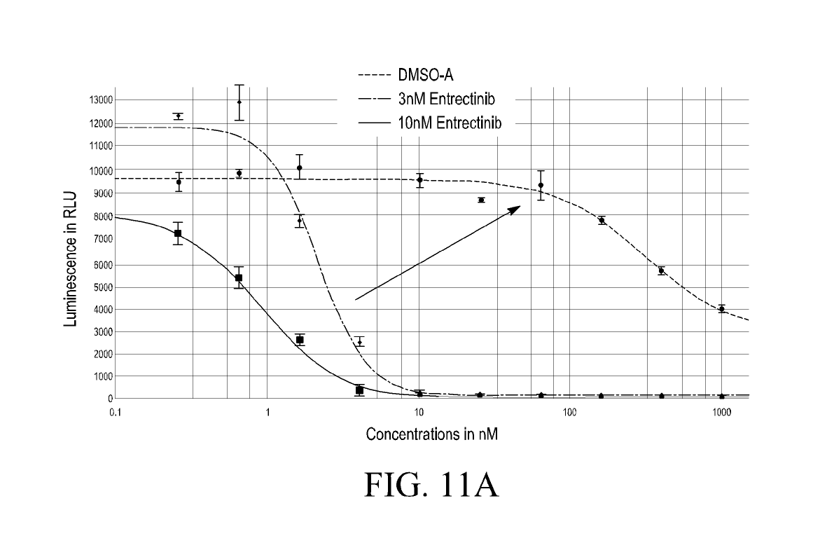

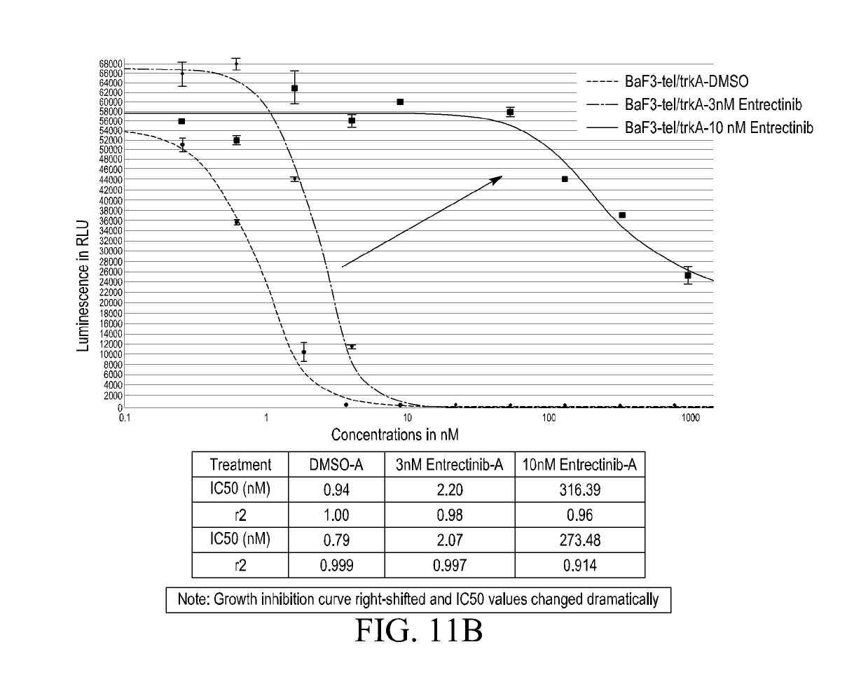

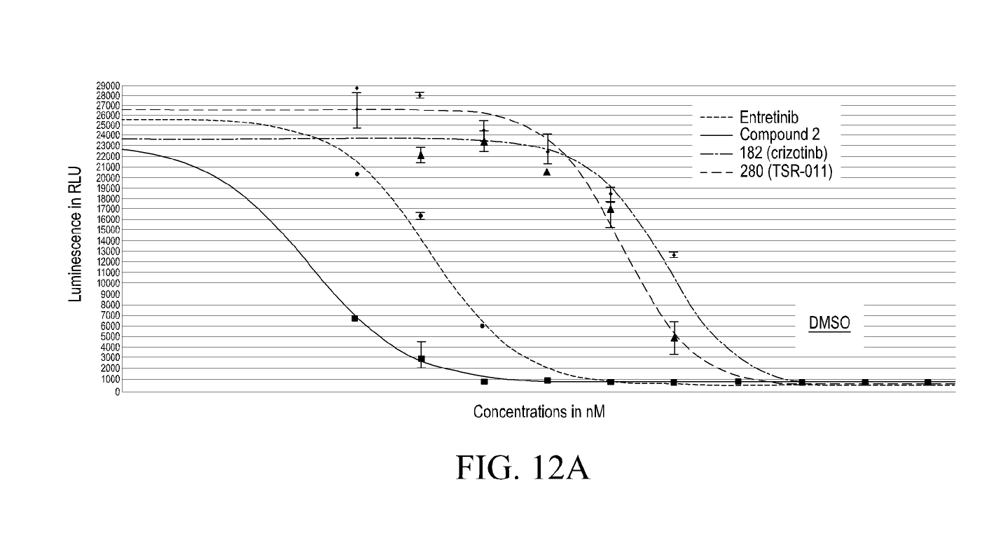

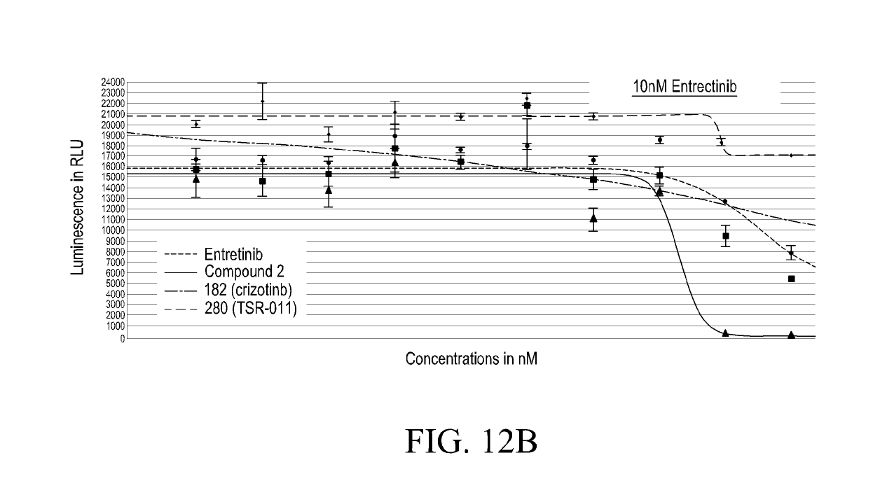

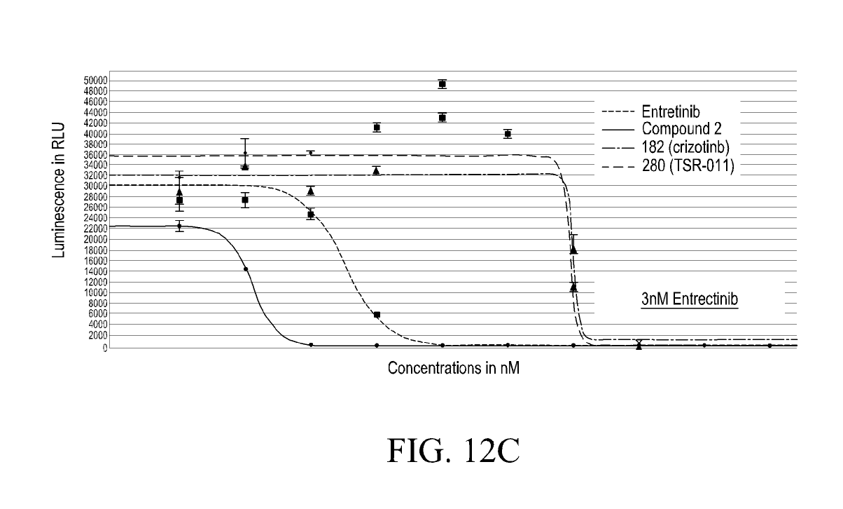

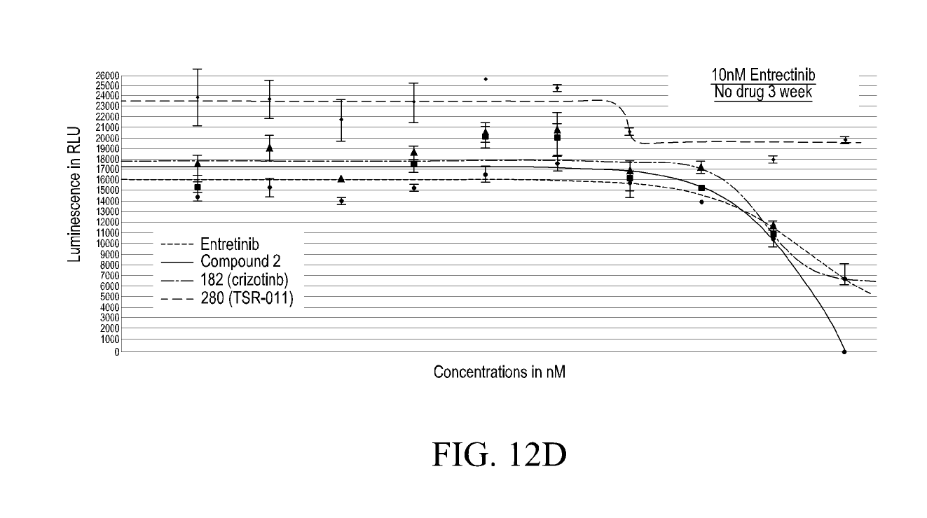

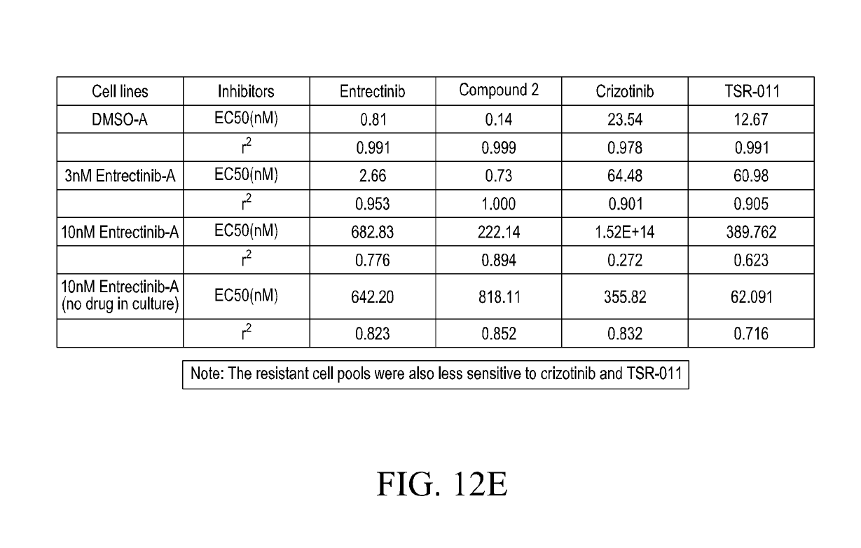

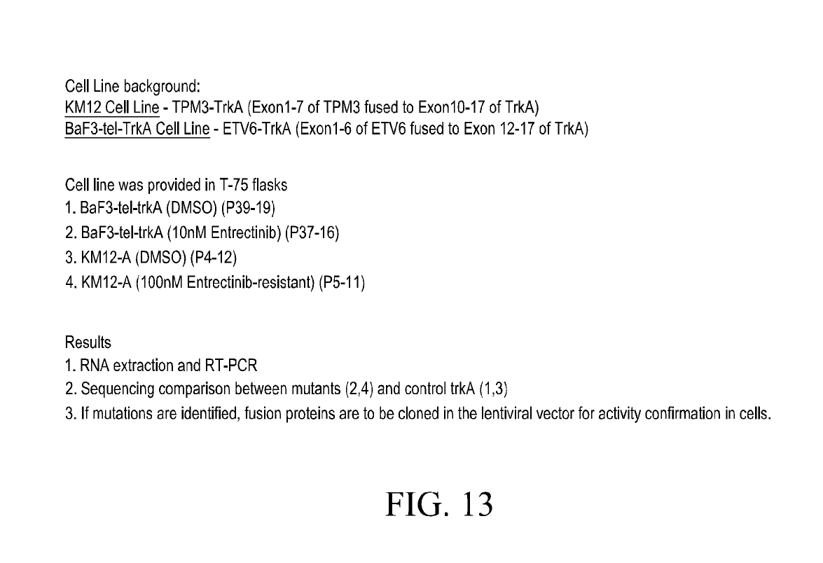

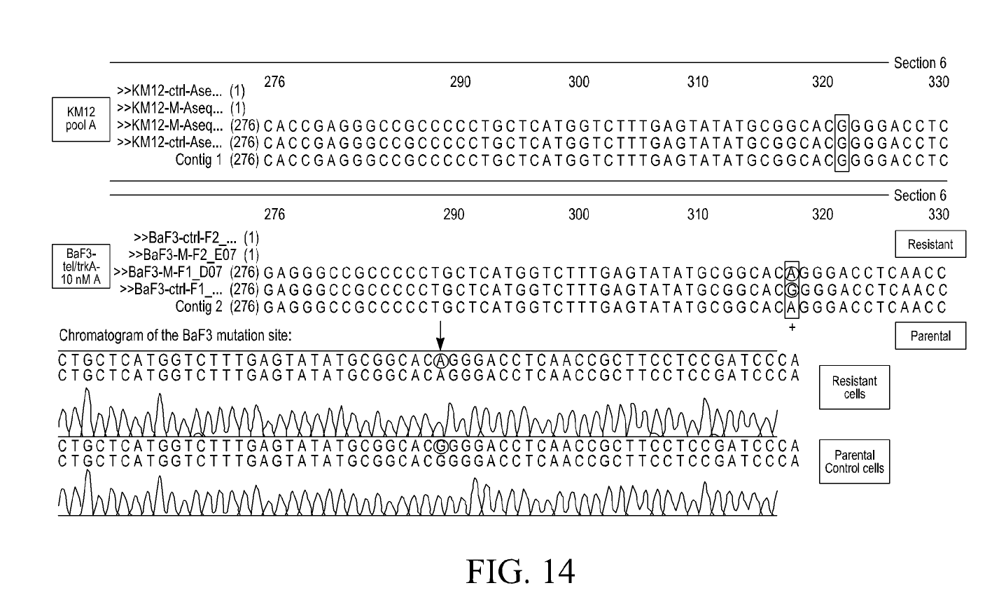

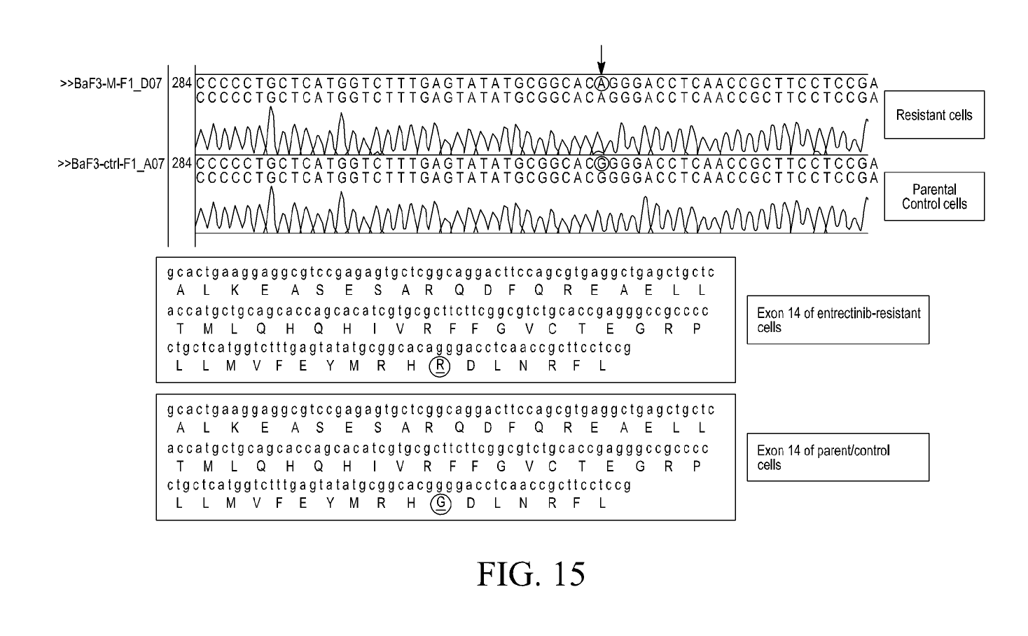

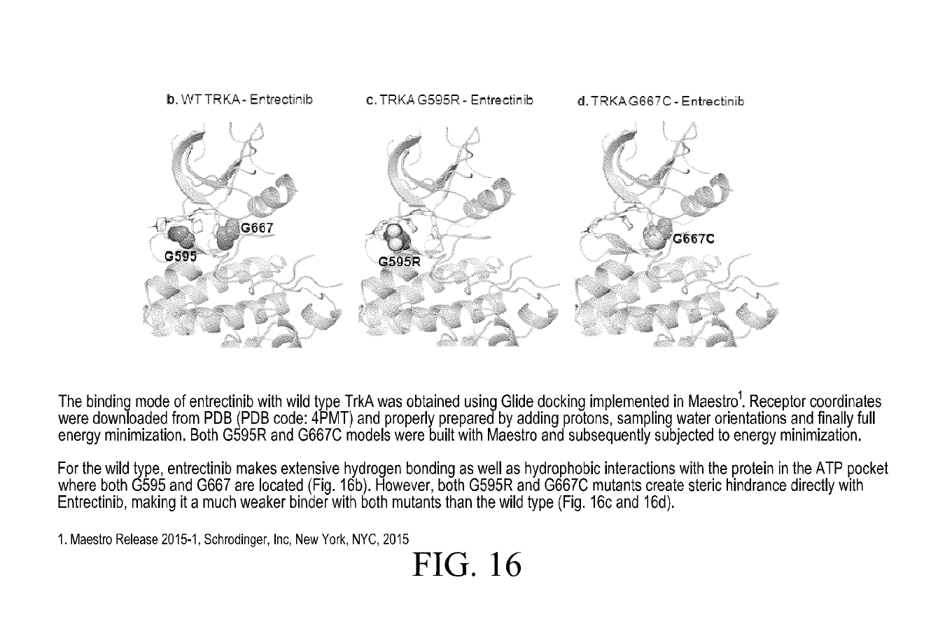

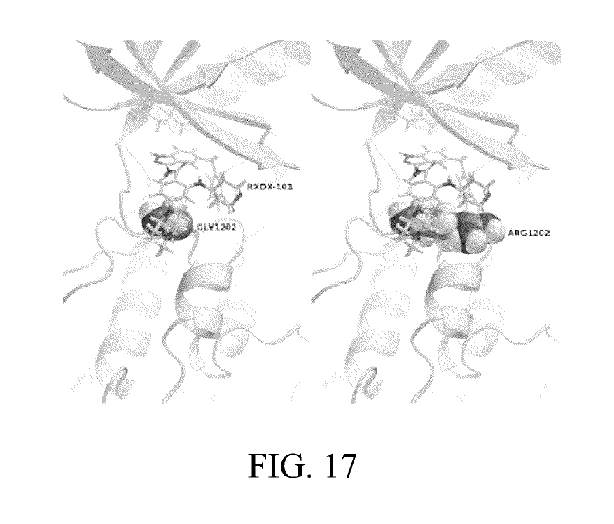

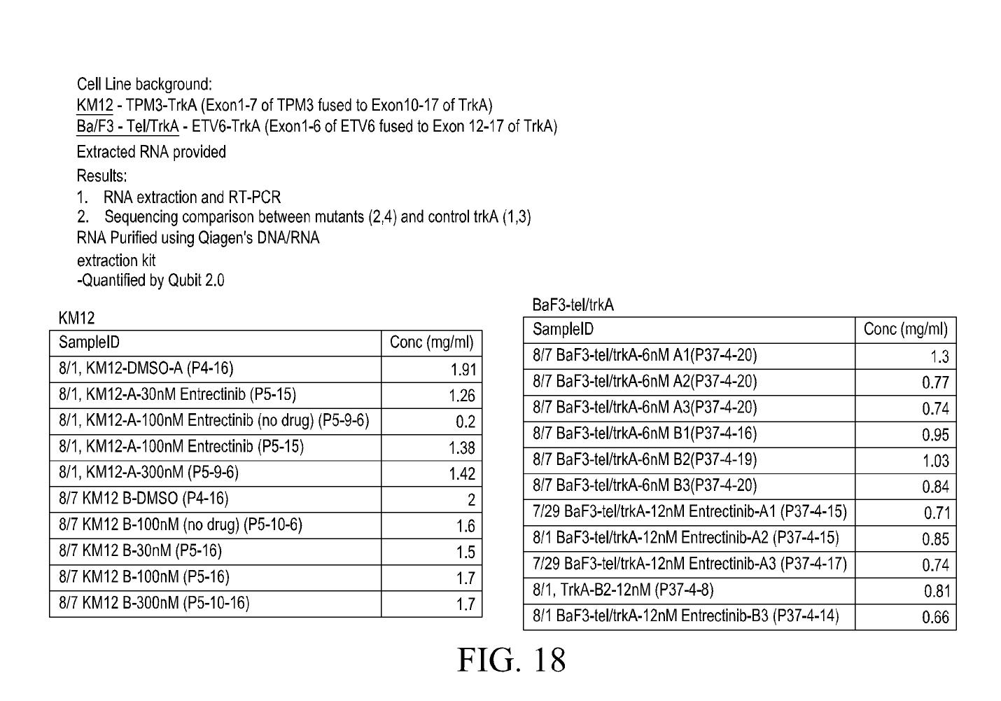

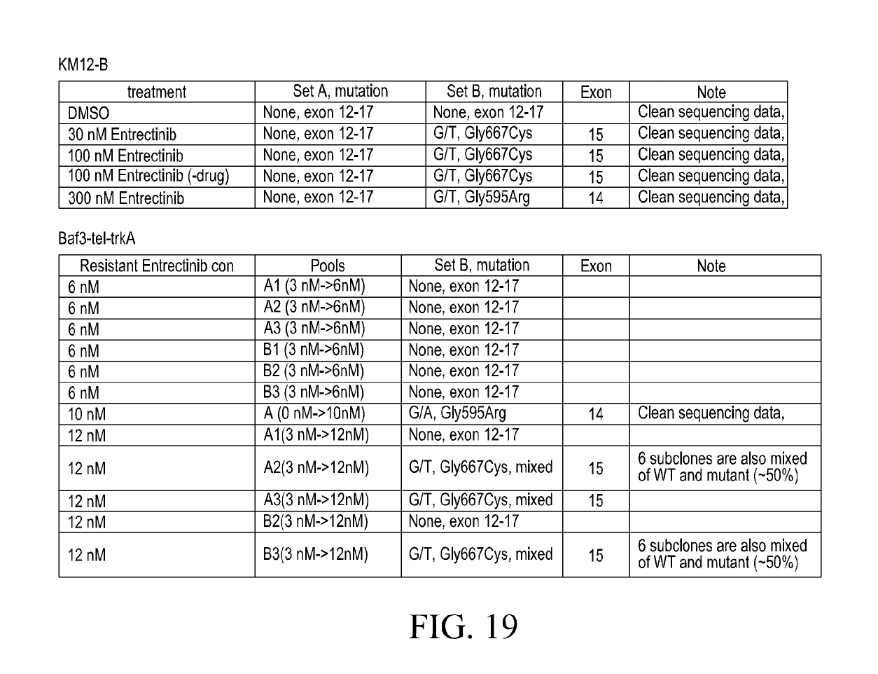

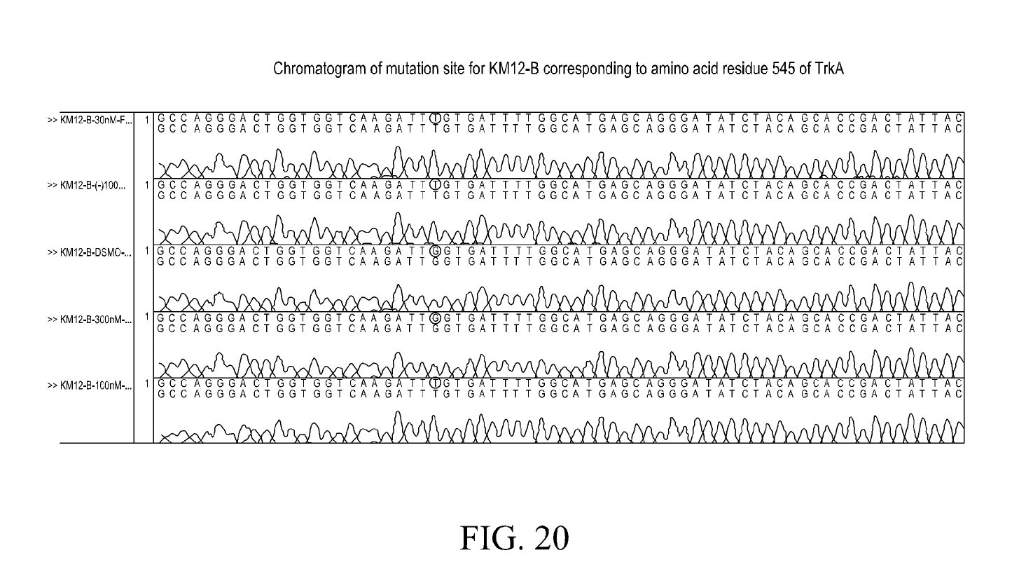

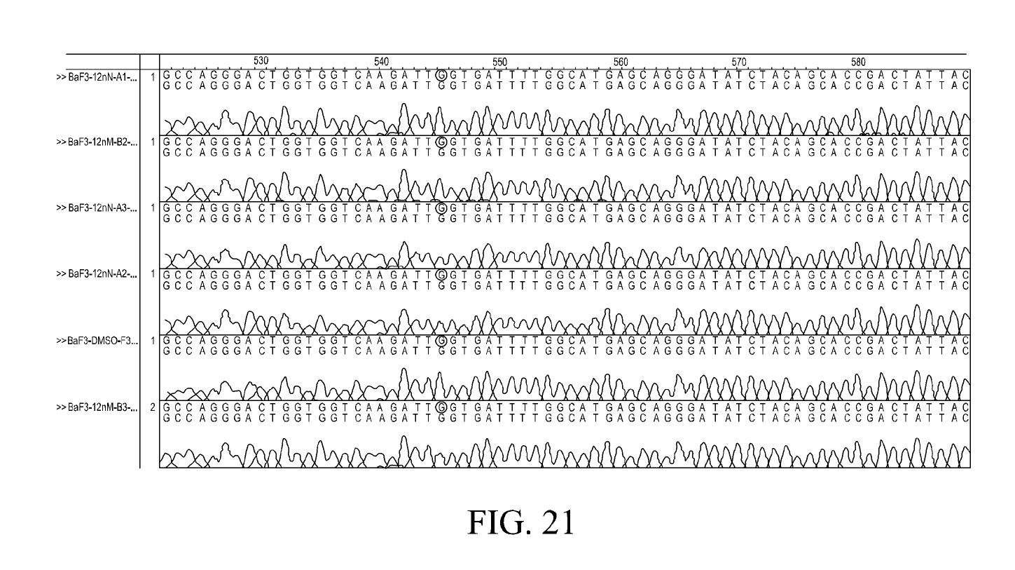

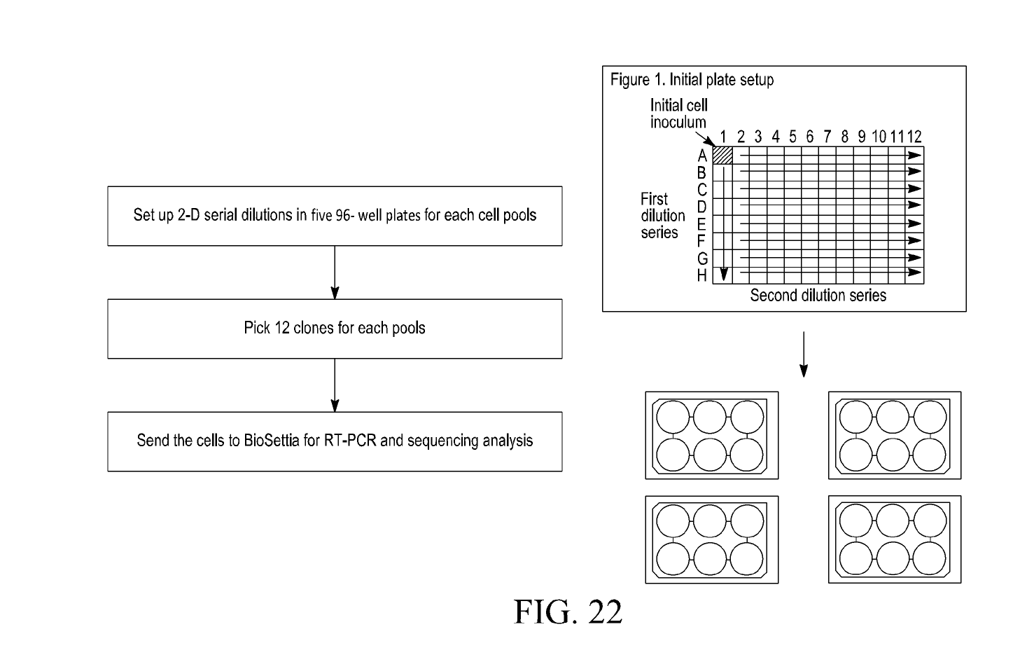

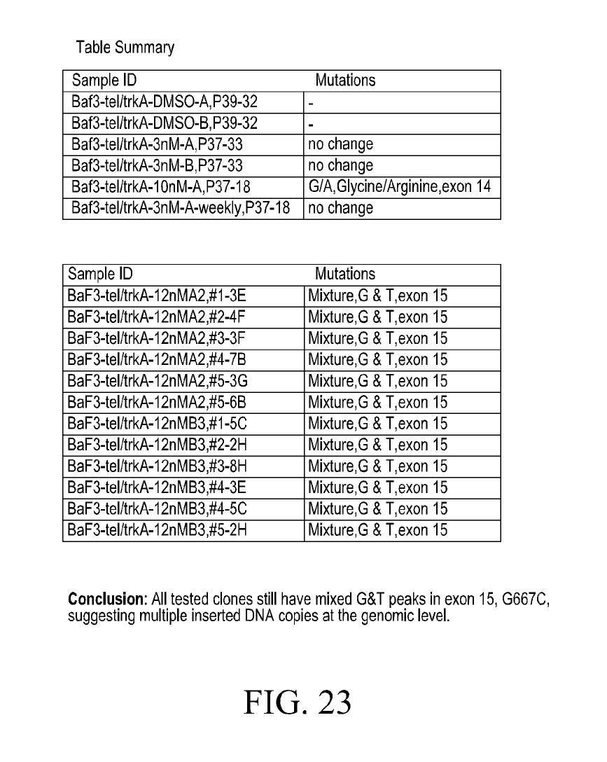

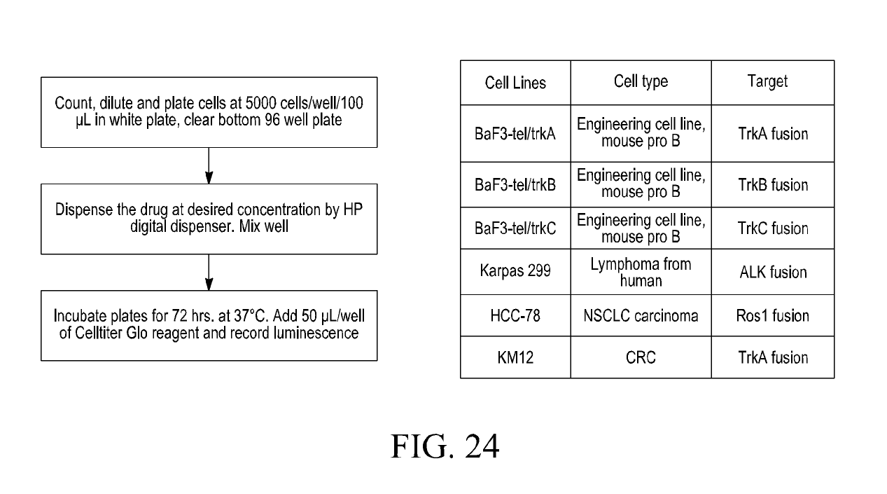

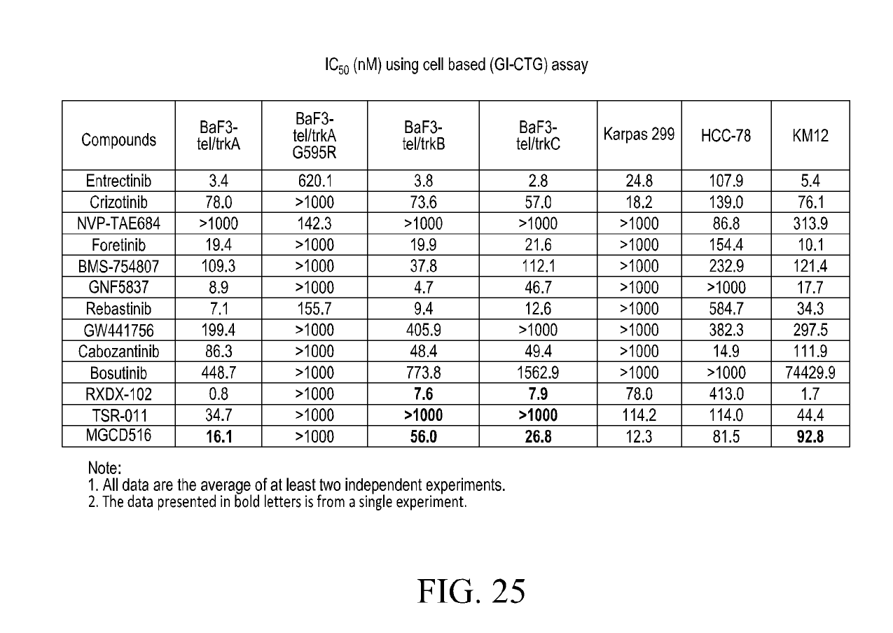

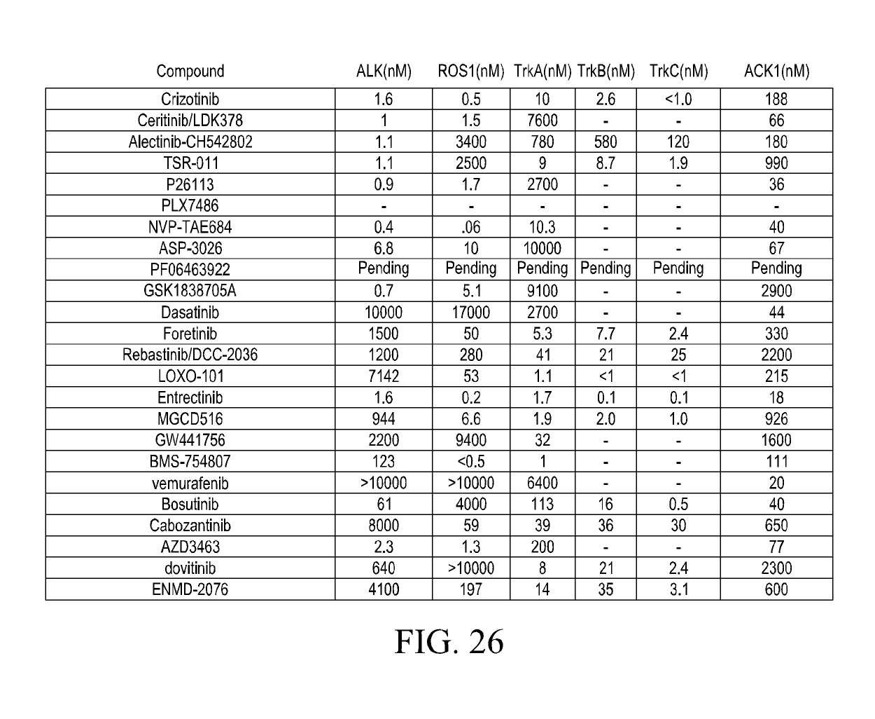

Parental Ba/F3-Tel/TrkA cells were treated with the RTK inhibitor entrectinib at treatment regimens as described in Example 4 above. Entrectinib-resistant Ba/F3-Tel/TrkA cells were isolated and subsequently characterized by using the procedures described in Example 4. Cell pools of 10 nM entrectinib-resistant Ba/F3-Tel/TrkA were established after 2-week selection (FIG. 9). Remarkably, as showed in FIGS. 11A and 11B, a 10 nM entrectinib-resistant Ba/F3-Tel/TrkA-10nMA cell pool displayed an IC50 which was >100 fold higher than that of the control parental line, indicating a drastic reduction of these cells' sensitivity to entrectinib. As showed at FIG. 15, these entrectinib-resistant Baf3-trkA (A) cells were found to harbor the same G667C and G595R mutations as discussed above in Example 4 with regard to the entrectinib-resistant KM12 cell lines. Additionally, as shown in FIG. 19, a different treatment of Ba/F3-Tel/TrkA at 12 nM Entrectinib resulted in a G667C change in multiple clones, which was also the same change identified in Entrectinib-KM12 resistant cells as discussed above in Example 4.

### Example 6

### Identification of Compounds Capable of Inhibiting Growth of Entrectinib-Resistant KM12 Cells and Entrectinib-Resistant Ba/F3-Tel/trkA Cells

This example describes a study performed to screen a number of chemical compounds for their ability to inhibit the proliferation of mutant KM12 and Ba/F3-tel/trkA cells harboring either G595R or G667C mutation, using the experimental procedure described in Example 2 above. Such compounds, once identified, would be useful in the treatment of cancer patients that have developed resistance to an inhibitor of a receptor tyrosine kinase. In this experiment, each of the following cell lines: Ba/F3-tel/trkA, Ba/F3-tel/trkA-10nMA (G595R), KM12-DMSO, KM12-30nM101-B (G667C), KM12-100nM101-B (G667C), KM12-300nM101-B (G595R) was screened against a number of chemical compounds. Exemplifications of such compounds are listed in TABLEs 2 and 8-9.

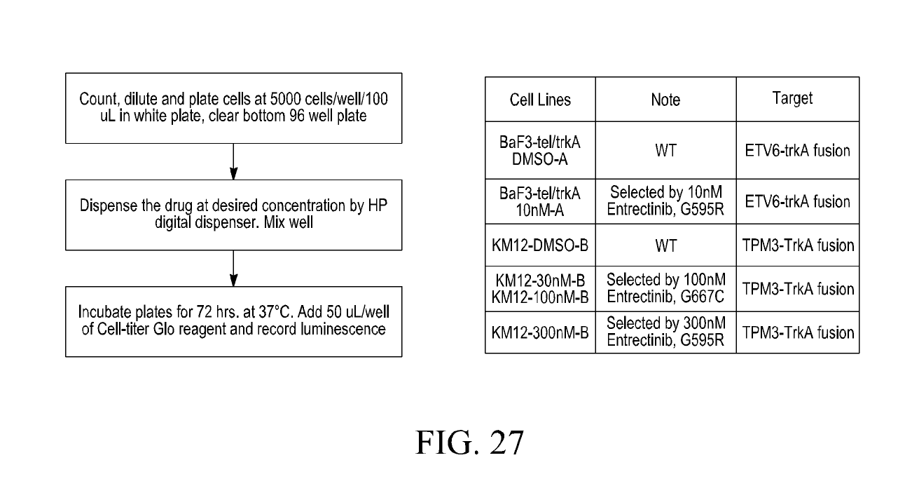

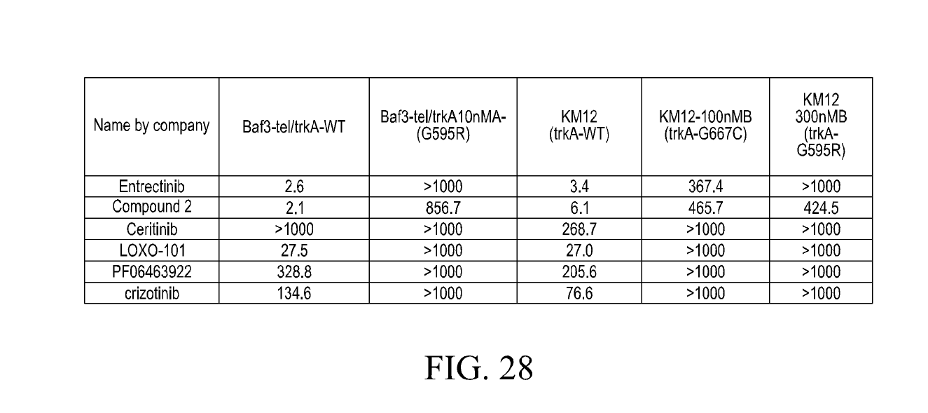

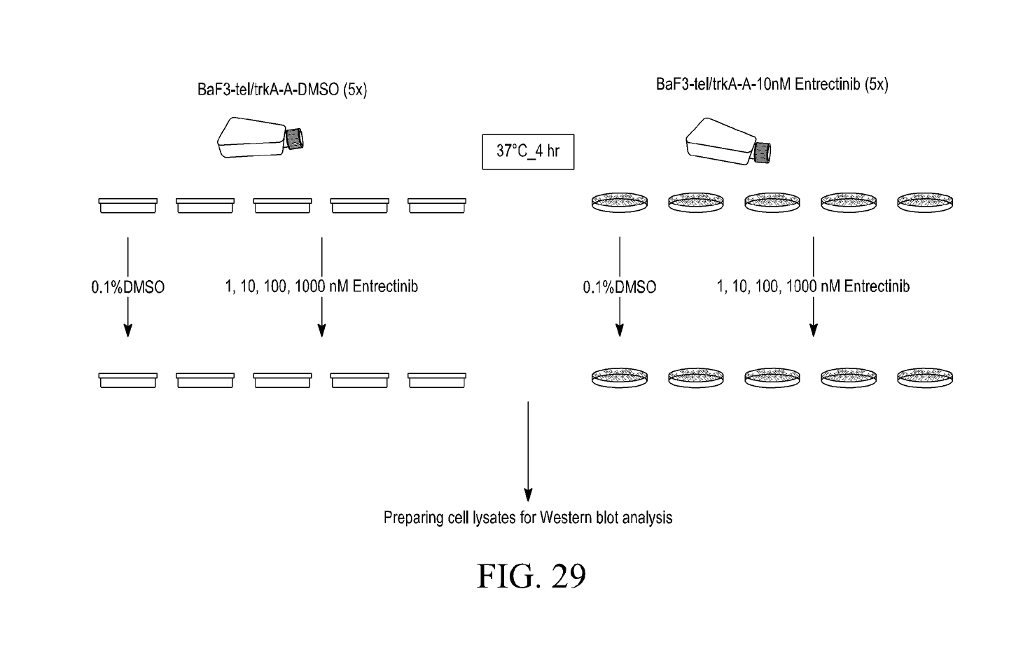

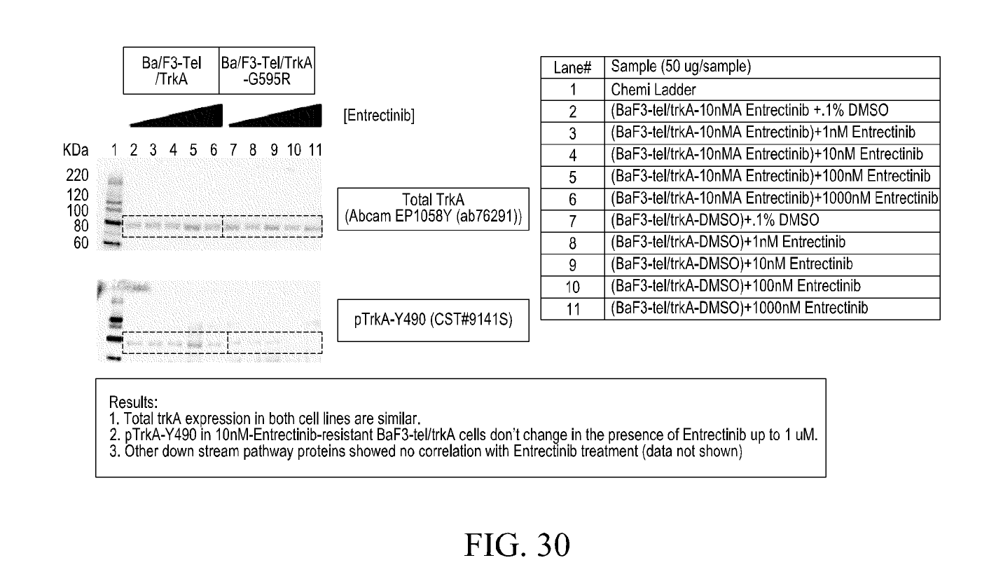

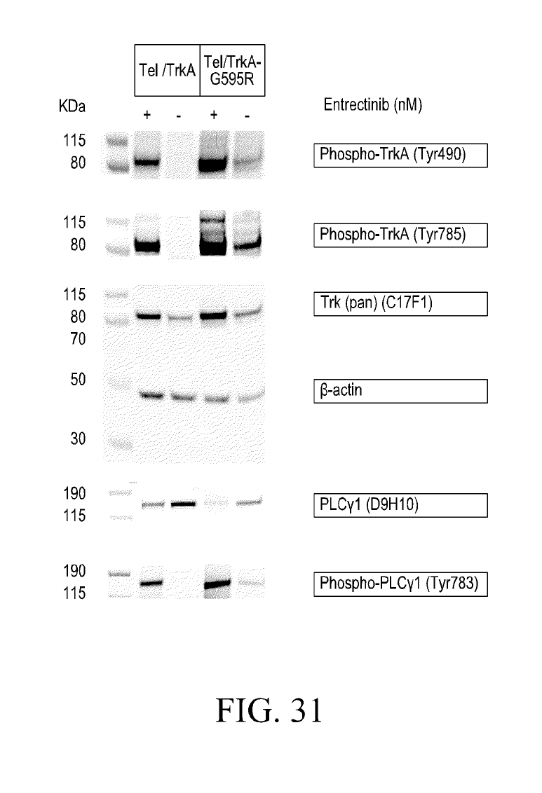

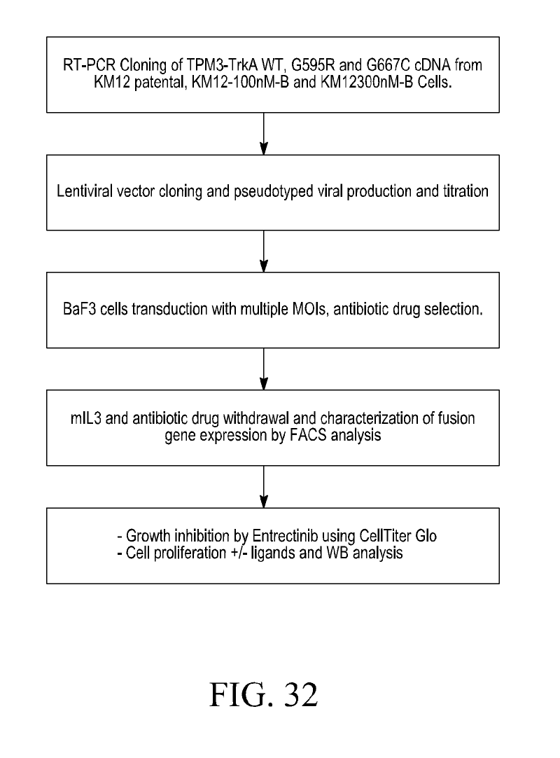

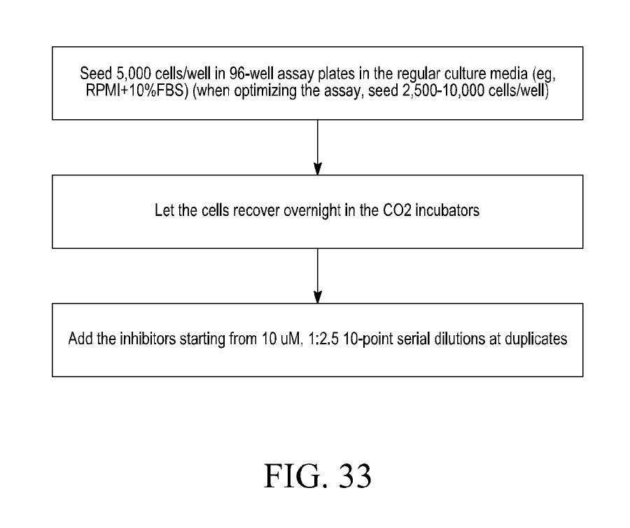

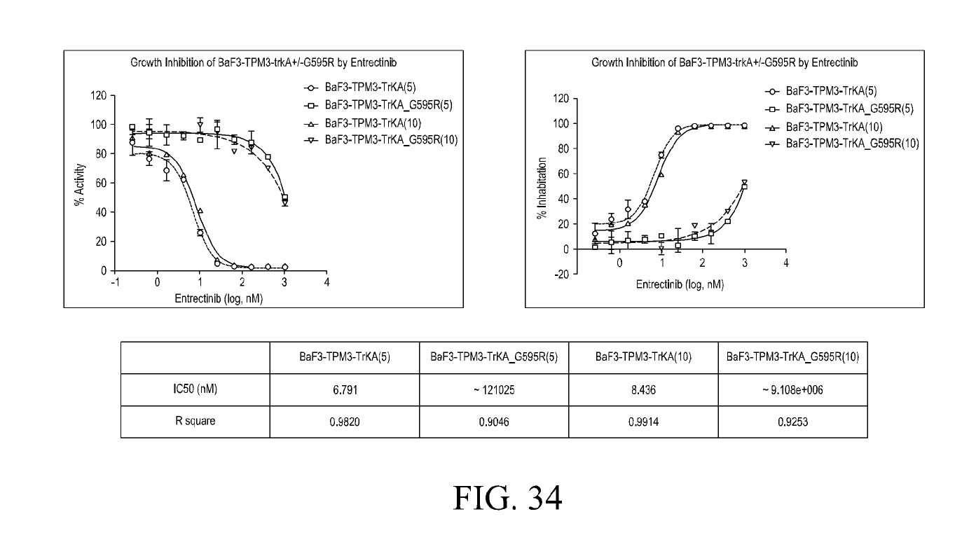

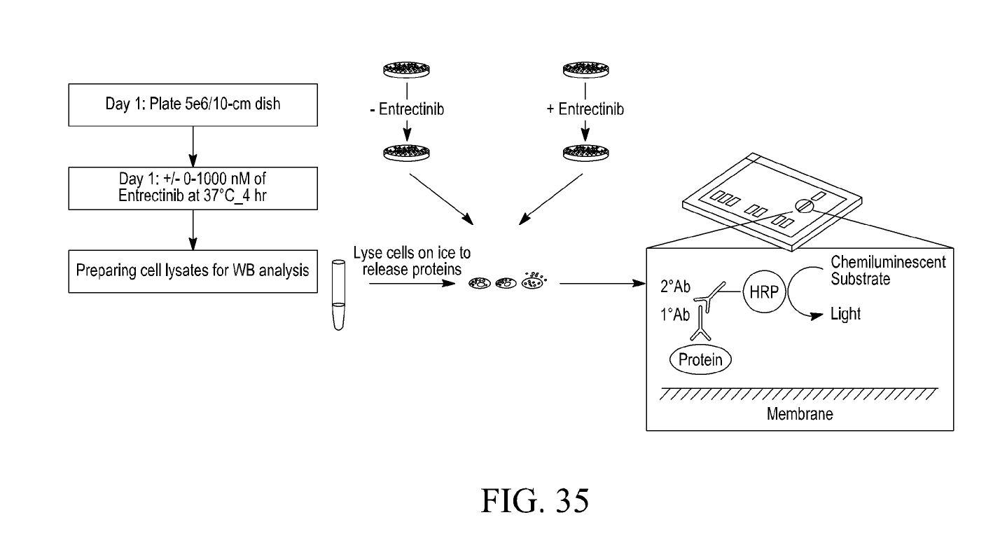

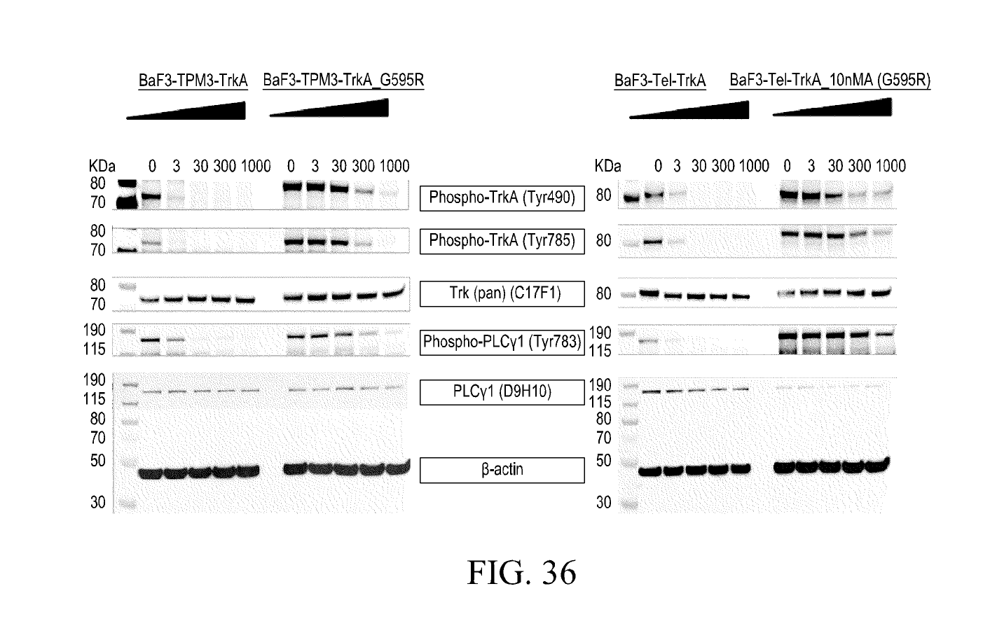

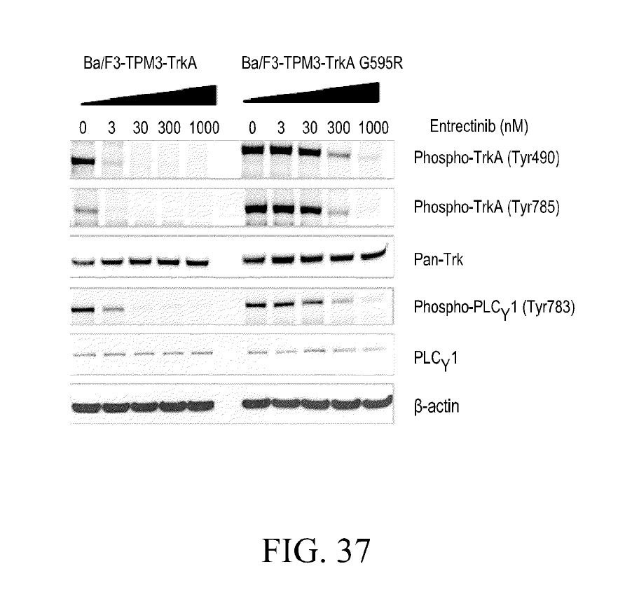

As showed in FIGS. 27 and 30, and TABLES 8-9 below, entrectinib, rebastinib, staurosporine, NVP-TAE684, and Compound 2 each showed significant inhibitory activity against mutant cells harboring the TrkA-G595C mutation or the TrkA-G667C mutation.

TABLE 8
IC50 values of six candidate compounds tested against parental KM12
and Ba/F3-tel/trkA cell lines (WT) and respective mutant cell lines
harboring the RKA-G595C mutation or the G595R mutation.

| | BaF3-tel/ | BaF3-tel/ | KM12-TPM3/ | KM12-TPM3/ | KM12-TPM3/ |
|---|---|---|---|---|---|
| Inhibitor | trkA_WT | trkA_G595R | trkA_WT | trkA_G667C | trkA_G595R |
| Entrectinib | 2.8 | >1 uM | 3.4 | 367.4 | >1 μM |
| Compound 2 | 2.3 | 846.2 | 6.1 | 465.7 | 424.5 |
| ceritinib | >1 μM | >1 μM | 268.7 | >1 μM | >1 μM |
| LOXO-101 | 33.0 | >1 μM | 27.0 | >1 μM | >1 μM |
| PF06463922 | 385.6 | >1 μM | 205.6 | >1 μM | >1 μM |
| crizotinib | 179.2 | 432.8 | 76.6 | >1 μM | >1 μM |
| (PF1066) Xalkori | | | | | |

TABLE 9
IC50 values of four candidate compounds tested against parental
Ba/F3-tel/trkA, Ba/F3-tel/trkB, Ba/F3-tel/trkC cell lines (WT) and
mutant BaF3-tel/trkA cell line harboring the G595R mutation.

| | IC50 (nM) | | | |
|---|---|---|---|---|
| | | BaF3-tel/ | | |
| Name by | BaF3-tel/ | trkA-10 | BaF3-tel/ | BaF3-tel/ |
| company | trkA | nMA(G595R) | trkB | trkC |
| entrectinib | 2.5 | 1099.0 | 8.7 | 5.5 |
| AZ-23 | 1.7 | 91.4 | 3.3 | 2.2 |
| K252a | 10.9 | 133.3 | 45.3 | 14.3 |
| Starausporine | 2.0 | 6.5 | 4.2 | 3.3 |

### Example 7

### Activities of Entrectinib and LOXO-101 in the Inhibition of Growth of Ba/F3 Cell Lines Expressing NTRK1 Wild-Type and Various Mutant NTRK1

This example describes a study performed to study the ability of the entrectinib and LOXO-101 compounds to inhibit the proliferation of wild-type and mutant Ba/F3-tel/trkA sells harboring various mutations, using the experimental procedure described in Example 2 above. In this experiment, each of entrectinib and LOXO-101 was screened against the mutants in TABLE 10.

TABLE 10
IC50 values of entrectinib and LOXO-101 tested against Ba/F3 Cell
Lines Expressing NTRK1 Wild-Type and Various Mutant NTRK1

| | NTRK1 | Entrectinib | LOXO-101 |
|---|---|---|---|
| Cell lines | Mutation | (nM) | (nM) |
| Ba/F3-LNMA-NTRK1 | | 2.3 | 11.9 |
| Ba/F3-LNMA-NTRK1-V573M | V573M | 24.2 | 621.9 |
| Ba/F3-LNMA-NTRK1-G667A | G667A | 5.1 | 74.4 |
| Ba/F3-LNMA-NTRK1-G667S | G667S | 14.6 | 197.7 |
| Ba/F3-LMNA-NTRK1 F589L | F589L | 9.7 | >1000 |
| Ba/F3-LNMA-NTRK1-G595R | G595R | >1000 | >1000 |
| Ba/F3-TPM3-NTRK1 | | 5.0 | 15.1 |
| Ba/F3-TPM3-NTRK1-G667C | G667C | 69.0 | >1000 |
| Ba/F3-TPM3-NTRK1-G595R | G595R | >1000 | >1000 |

### Example 8

### Activities of Entrectinib, LOXO-101, and Staurosporine in the Inhibition of Growth of Ba/F3 Cell Lines Expressing NTRK1 Wild-Type and Various Mutant NTRK1

This example describes a study performed to study the ability of the entrectinib, LOXO-101, and staurosporine compounds to inhibit the proliferation of wild-type and mutant Ba/F3-tel/trkA cells harboring various mutations, using the experimental procedure described in Example 2 above. In this experiment, each of entrectinib, LOXO-101, and staurosporine was screened against the mutants in TABLE 11.

TABLE 11
IC50 values of entrectinib, LOXO-101 and staurosporine
tested against Ba/F3 Cell Lines Expressing NTRK1
Wild-Type and Various Mutant NTRK1

| | | | Stauro- |
|---|---|---|---|
| | Entrectinib | LOXO-101 | sporine |
| Cell Lines | (nM) | (nM) | (nM) |
| BaF3-LMNA-NTRK1 | 2.4 | 15.4 | 2.2 |
| BaF3-LMNA-NTRK1-F589L | 2.9 | >1000 | 1.6 |
| BaF3-LMNA-NTRK1-G667A | 3.8 | 61.4 | 0.6 |
| BaF3-LMNA-NTRK1-G595R | >1000 | >1000 | 3.9 |

All of the references disclosed herein, including but not limited to journal articles, textbooks, patents and patent applications, are hereby incorporated by reference for the subject matter discussed herein and in their entireties. However, no admission is made that any reference cited herein constitutes prior art. Throughout this disclosure, various information sources are referred to and incorporated by reference. The information sources include, for example, scientific journal articles, patent documents, textbooks, and World Wide Web browser-inactive page addresses. The reference to such information sources is solely for the purpose of providing an indication of the general state of the art at the time of filing. While the contents and teachings of each and every one of the information sources can be relied on and used by one of skill in the art to make and use the embodiments disclosed herein, any discussion and comment in a specific information source should no way be considered as an admission that such comment was widely accepted as the general opinion in the field.

The discussion of the general methods given herein is intended for illustrative purposes only. It is not intended to be exhaustive or to limit the disclosure. Individual aspects or features of a particular embodiment are generally not limited to that particular embodiment, but, where applicable, are interchangeable and can be used in a selected embodiment, even if not specifically shown or described. It is expressly contemplated that any aspect or feature of the present disclosure can be combined with any other aspect, features, or combination of aspects and features disclosed herein. Other alternative methods and embodiments will be apparent to those of skill in the art upon review of this disclosure, and are to be included within the spirit and purview of this application.

1-396. (canceled)

397. A method for treating a patient having a cancer tumor, comprising
a) determining the presence of a nucleic acid encoding a mutated Trk protein in a tumor sample from said patient, wherein said mutated Trk protein comprises at least one mutation at an amino acid position selected from:
i. V573, F589, G595 and G667 of the TrkA polypeptide set forth in SEQ ID NO: 1;
ii. V619, F633, G639 and G709 of the TrkB polypeptide set forth in SEQ ID NO: 3; and
iii. V603, F617, G623 and G696 of the TrkC polypeptide set forth in SEQ ID NO: 5; and
b) administering to said patient a Trk-inhibiting compound.

398. The method of claim 397, wherein said one or more amino acid mutations is at a position corresponding to amino acid residue V573 of the TrkA polypeptide.

399. The method of claim 398, wherein said one or more amino acid mutations is a Val-to-Met substitution (V573M).

400. The method of claim 397, wherein said one or more amino acid mutations is at a position corresponding to amino acid residue F589 of the TrkA polypeptide.

401. The method of claim 400, wherein said one or more amino acid mutations is a Phe-to-Leu substitution (F598L).

402. The method of claim 397, wherein said one or more mutations is at a position corresponding to amino acid residue G595 of the TrkA polypeptide.

403. The method of claim 402, wherein said one or more mutations is a Gly-to-Arg substitution (G595R).

404. The method of claim 397, wherein said one or more mutations is at a position corresponding to amino acid residue G667 of the TrkA polypeptide.

405. The method of claim 404, wherein said one or more mutations is a Gly-to-Cys substitution (G667C).

406. The method of claim 404, wherein said one or more amino acid mutations is a Gly-to-Ala substitution (G667A).

407. The method of claim 404, wherein said one or more amino acid mutations is a Gly-to-Ser substitution (G667S).

408. The method of claim 397, wherein said one or more amino acid mutations is at a position corresponding to amino acid residue V619 of the TrkB polypeptide.

409. The method of claim 408, wherein said one or more amino acid mutations is a Val-to-Met substitution (V619M).

410. The method of claim 397, wherein said one or more amino acid mutations is at a position corresponding to amino acid residue F633 of the TrkB polypeptide.

411. The method of claim 410, wherein said one or more amino acid mutations is a Phe-to-Leu substitution (F633L).

412. The method of claim 397, said one or more mutations is at a position corresponding to amino acid residue G639 of the TrkB polypeptide.

413. The method of claim 412, wherein said one or more mutations is a Gly-to-Arg substitution (G639R).

414. The method of claim 397, wherein said one or more mutations is at a position corresponding to amino acid residue G709 of the TrkB polypeptide.

415. The method of claim 414, wherein said one or more mutations is a Gly-to-Cys substitution (G709C).

416. The method of claim 414, wherein said one or more amino acid mutations is a Gly-to-Ala substitution (G709A).

417. The method of claim 414, wherein said one or more amino acid mutations is a Gly-to-Ser substitution (G709S).

418. The method of claim 397, wherein said one or more amino acid mutations is at a position corresponding to amino acid residue V603 of the TrkC polypeptide.

419. The method of claim 418, wherein said one or more amino acid mutations is a Val-to-Met substitution (V603M).

420. The method of claim 397, wherein said one or more amino acid mutations is at a position corresponding to amino acid residue F617 of the TrkC polypeptide.

421. The method of claim 420, wherein said one or more amino acid mutations is a Phe-to-Leu substitution (F617L).

422. The method of claim 397, wherein said one or more mutations is at a position corresponding to amino acid residue G623 of the TrkC polypeptide.

423. The method of claim 422, wherein said one or more mutations is a Gly-to-Arg substitution (G623R).

424. The method of claim 397, wherein said one or more mutations is at a position corresponding to amino acid residue G696 of the TrkC polypeptide.

425. The method of claim 424, wherein said one or more mutations is a Gly-to-Cys substitution (G696C).

426. The method of claim 424, wherein said one or more amino acid mutations is a Gly-to-Ala substitution (G696A).

427. The method of claim 424, wherein said one or more amino acid mutations is a Gly-to-Ser substitution (G696S).

428. The method of any claim 397, wherein said cancer is selected from anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, papillary thyroid cancer, or any combination thereof.
